(19) United States
(12) Patent Application Publication
(10) Pub. No.: US 2011/0118339 A1
(43) Pub. Date: May 19, 2011

(54) CHEMICALLY MODIFIED OLIGONUCLEOTIDES AND USES THEREOF

(75) Inventors: Muthiah Manoharan, Cambridge, MA (US); Kallanthottathil G. Rajeev, Cambridge, MA (US)

(73) Assignee: ALNYLAM PHARMACEUTICALS, INC., Cambridge, MA (US)

(21) Appl. No.: 12/863,359

(22) PCT Filed: Jan. 16, 2009
(86) PCT No.: PCT/US09/31259

§ 371 Date: Jan. 18, 2011

Related U.S. Application Data
(60) Provisional application No. 61/022,301, filed on Jan. 18, 2008.

(51) Int. Cl. A61K 31/7105; A61P 35/00

(52) U.S. Cl. 514/44 R

(57) ABSTRACT

This invention relates generally to chemically modified oligonuceotides useful for augmenting activity of microRNAs and pre-microRNAs. E.g., the invention relates to single stranded chemically modified oligonuceotides for augmenting microRNA and pre-microRNA expression and to methods of making and using the modified oligonucleotides.

### RELATED APPLICATIONS

This application claims the benefit of priority to U.S. Provisional Patent Application Ser. No. 61/022,301, filed Jan. 18, 2008.

### TECHNICAL FIELD

This invention relates generally to agents, e.g., chemically modified oligonucleotides useful for upregulating expression of microRNAs. More particularly, the invention relates to single stranded, double stranded, partially double stranded and hairpin structured chemically modified oligonucleotides. In preferred embodiments the agents augment the effect of an endogenous microRNA on gene expression and to methods of making and using the agents.

### BACKGROUND

A variety of nucleic acid species are capable of modifying gene expression. These include antisense RNA, siRNA, microRNA, RNA and DNA aptamers, and decoy RNAs. Each of these nucleic acid species can inhibit target nucleic acid activity, including gene expression.

MicroRNAs (miRNAs) are a class of 18-24 nt non-coding RNAs (ncRNAs) that exist in a variety of organisms, including mammals, and are conserved in evolution. miRNAs are processed from hairpin precursors of 70 nt (pre-miRNA) which are derived from primary transcripts (pri-miRNA) through sequential cleavage by the RNAse III enzymes drosha and dicer. miRNAs can be encoded in intergenic regions, hosted within introns of pre-mRNAs or within ncRNA genes. Many miRNAs also tend to be clustered and transcribed as polycistrons and often have similar spatial temporal expression patterns. MiRNAs have been found to have roles in a variety of biological processes including developmental timing, differentiation, apoptosis, cell proliferation, organ development, and metabolism.

### SUMMARY

The present invention is based in part on the discovery that activity of endogenous microRNAs (miRNAs) or pre-microRNAs (pre-miRNAs) can be augmented by an agent such as an oligonucleotide agent or small molecule agent described herein, e.g., through systemic administration of the agent, as well as by parenteral administration of such agents. Embodiments of the invention provide specific compositions and methods that are useful in augmenting miRNA or pre-miRNA activity levels, in e.g., a mammal, such as a human. In particular, the present invention provides specific compositions and methods that are useful for enhancing activity levels of miRNAs, e.g., miR-122, miR-16, miR-192, miR-194, miR-141, mRR-143, miR-181, miR-181a, miR-181e, miR-192, miR-194, miR-200c, miR-206, miR-1, miR-205, miR-16, miR ebv-BHRF1-1, miR ebv-BHRF1-2, miR ebv-BHRF12-1, miR kshv-K3, miR kshv-K4-3p, miR kshv-mir-K2, miR kshv-mir-K5, miR kshv-mir-K6-3p, miR kshv-mir-K7, miR kshv-mir-K11, miR-31, miR-196, miR-215, miR-155, miR-142-5p, miR-142-3p, miR-143, Hsa-mir-146a, Hsa-mir-146b, mCMV-miR-01-1, mCMV-miR-0′-2, mCMV-miR-23-1, mCMV-miR-23-2, mCMV-miR-44-1, miR-133, miR-133b, miR-124, miR-126, miR-126-3p, miR-126-5p, miR-21, miR-22, miR-122, miR-33.

In one aspect, the invention features oligonucleotide agents called supermirs. Supermirs are single stranded, double stranded, partially double stranded and hairpin structured chemically modified oligonucleotides that have a sequence substantially identical to an endogenous microRNA sequence, and that target the same RNA as the endogenous miRNA. FIGS. 1-3 provides representative structures of supermirs.

An olignucleotide agent featured in the invention, e.g., a supermir, consists essentially of or includes at least 12 or more contiguous nucleotides substantially identical to an endogenous miRNA, and more particularly includes 12 or more contiguous nucleotides substantially complementary to a target sequence of an miRNA or pre-miRNA nucleotide sequence. Preferably, an oligonucleotide agent featured in the invention includes a nucleotide sequence sufficiently complementary to hybridize to a miRNA target sequence of about 12 to 25 nucleotides, preferably about 15 to 23 nucleotides. More preferably, the oligonucleotide agent includes a sequence that differs by no more than 1, 2, or 3 nucleotides from a sequence shown in Table 1, and in one embodiment, the oligonucleotide agent is an agent shown in Table 2. In one embodiment, the agent includes a non-nucleotide moiety, e.g., a cholesterol moiety. The non-nucleotide moiety can be attached, e.g., to the 3′ or 5′ end of the oligonucleotide agent. In a preferred embodiment, a cholesterol moiety is attached to the 3′ end of the oligonucleotide agent.

An oligonucleotide agent featured herein, e.g., a supermir can be modified, for example, to provide increased stability against nucleolytic degradation. Exemplary modifications include a modification of the nucleotide backbone such as modification of the phosphate linker or replacement of the phosphate linker; modification of the sugar moiety such as modification of the 2′ hydroxyl on the ribose; replacement of the sugar moiety such as ribose or deoxyribose with a different chemical structure such as a PNA structure; or modification of the nucleobase for example modification to a universal base or G-clamp. In some embodiments, the oligonucleotide agent includes a phosphorothioate in at least the first, second, or third internucleotide linkage at the 5′ or 3′ end of the nucleotide sequence. In one in embodiment, the oligonucleotide agent includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (T-O-DMAP), 2′-O-dimethylaminoethyloxyethyl (2′-O-DMAEOE), or T-O—N-methylacetamido (2′-O-NMA). In a particularly preferred embodiment, the oligonucleotide agent includes at least one 2′-O-methyl-modified nucleotide, and in some embodiments, all of the nucleotides of the oligonucleotide agent include a 2′-O-methyl modification.

The oligonucleotide agent can be further modified so as to be attached to a ligand, for example, a ligand selected to improve stability, distribution or cellular uptake of the agent, e.g., cholesterol or folate. Exemplary lipophilic ligands include a cholesterol; a bile acid; and a fatty acid (e.g., lithocholic-oleyl, lauroyl, docosnyl, stearoyl, palmitoyl, myristoyl, oleoyl, linoleoyl). In some preferred embodiments, the oligonucleotide agent is combined with a targeting agent such as a folate moiety.

The oligonucleotide agent can further be in isolated form or can be part of a pharmaceutical composition used for the methods described herein, particularly as a pharmaceutical composition formulated for parental administration. The pharmaceutical compositions can contain one or more oligonucleotide agents, and in some embodiments, will contain two or more oligonucleotide agents, each one directed to a different miRNA.

An oligonucleotide agent, e.g., a supermir, that is substantially identical to a nucleotide sequence of an miRNA can be delivered to a cell or a human to augment the activity of an endogenous miRNA, or activity of a target mRNA that hybridizes to the endogenous miRNA, is linked to a disease or disorder. In one embodiment, an oligonucleotide agent featured in the invention includes a nucleotide sequence that is substantially identical to miR-122 (see Table 1), which hybridizes to numerous RNAs, including aldolase A mRNA, N-myc downstram regulated gene (Ndrg3) mRNA, IQ motif containing GTPase activating protein-1 (Iqgap1) mRNA, HMG-CoA-reductase (Hmgcr) mRNA, and citrate synthase mRNA and others. In a preferred embodiment, the oligonucleotide agent that is substantially identical to miR-122 is discosled herein. Aldolase A has been shown to be overexpressed in different cancers, including lung cancer and breast cancer, and is overexpressed in adenocarcinomas of various different tissues origins. Thus ab agent described herein which agonizes miR-122, e.g., a single stranded supermir that is substantially identical to miR-122, can be administered as a therapeutic composition to a subject having or at risk for developing a disorder characterized by unwanted dell proliferation, e.g., cancer, e.g., lung cancer or breast cancer. Thus a human who has or who is diagnosed as having any of these disorders or symptoms is a candidate to receive treatment with an oligonucleotide agent that is substantially identical to miR-122.

In some embodiments, an oligonucleotide agent featured in the invention has a nucleotide sequence that is substantially identical to miR-16, miR-192, miR-194, miR-141, mRR-143, miR-181, miR-181a, miR-181c, miR-192, miR-194, miR-200c, R-206, miR-1, miR-205, miR-16, miR ebv-BHRF1-1, miR ebv-BHRF1-2, miR ebv-BHRF12-1, miR kshv-K3, miR kshv K4-3p, miR kshv-mir-K2, miR kshv-mir-K5, miR kshv-mir-K6-3p, miR kshv-mir-K7, miR kshv-mir-K11, miR-31, miR-196, miR-215, miR-155, miR-142-5p, miR-142-3p, miR-143, Hsa-mir-146a, Hsa-mir-146b, mCMV-miR-01-1, mCMV-miR-01-2, mCMV-miR-23-1, mCMV-miR-23-2, mCMV-miR-44-1, miR-133, miR-133b, miR-124, miR-126, miR-126-3p, miR-126-5p, miR-21, miR-22, miR-122, or miR-33.

In one aspect, the invention features a method of augmenting the activity levels of an miRNA or pre-miRNA in a cell of a subject, e.g., a human subject. The method includes the step of administering an oligonucleotide agent to the subject, where the oligonucleotide agent is substantially single-stranded and includes a sequence that is substantially complementary to 12 to 23 contiguous nucleotides, and preferably 15 to 23 contiguous nucleotides, of a target sequence of an miRNA or pre-miRNA nucleotide sequence. Preferably, the target sequence differs by no more than 1, 2, or 3 nucleotides from a microRNA or pre-microRNA sequence, such as a microRNA sequence shown in Table 1.

In one embodiment, the methods featured in the invention are useful for augmenting the activity level of an endogenous miRNA (e.g., miR-122, miR-16, miR-192, miR-194, miR-141, mRR-143, miR-181, miR-181a, miR-181c, miR-200c, miR-206, miR-1, miR-205, miR ebv-BHRF1-1, miR ebv-BHRF1-2, miR ebv-BHRF12-1, miR kshv-K3, miR kshv-K4-3p, miR kshv-mir-K2, miR kshv-mir-K5, miR kshv-mir-K6-3p, miR kshv-mir-K7, miR kshv-mir-K11, miR-31, miR-196, miR-215, miR-155, miR-142-5p, miR-142-3p, miR-143, Hsa-mir-146a, Hsa-mir-146b, mCMV-miR-01-1, mCMV-miR-01-2, mCMV-miR-23-1, mCMV-miR-23-2, mCMV-miR-44-1, miR-133, miR-133b, miR-124, miR-126, miR-126-3p, miR-126-5p, miR-21, miR-22, miR-122, or miR-33) or pre-miRNA in a cell, e.g, in a cell of a subject, such as a human subject. Such methods include contacting the cell with an oligonucleotide agent described herein for a time sufficient to allow uptake of the oligonucleotide agent into the cell.

in another aspect, the invention features a pharmaceutical composition including an oligonucleotide agent described herein, and a pharmaceutically acceptable carrier. In a preferred embodiment, the oligonucleotide agent included in the pharmaceutical composition includes a sequence that is substantially identical to miR-122, miR-16, miR-192, miR-194, miR-141, mRR-143, miR-181, miR-181a, miR-181c, miR-200c, miR-206, miR-1, miR-205, miR ebv-BHRF1-1, miR ebv-BHRF1-2, miR ebv-BHRF12-1, miR kshv-K3, miR kshv-K4-3p, miR kshv-mir-K2, miR kshv-mir-K5, miR kshv-mir-K6-3p, miR kshv-mir-K7, miR kshv-mir-K11, miR-31, miR-196, miR-215, miR-155, miR-142-5p, miR-142-3p, miR-143, Hsa-mir-146a, Hsa-mir-146b, mCMV-miR-01-1, mCMV-miR-01-2, mCMV-miR-23-1, mCMV-miR-23-2, mCMV-miR-44-1, miR-133, miR-133b, miR-124, miR-126, miR-126-3p, miR-126-5p, miR-21, miR-22, or miR-33.

In another aspect the invention features a method of augmenting the activity level of an miRNA (e.g., miR-122, miR-16, miR-192, miR-194, miR-141, mRR-143, miR-181, miR-181a, miR-181c, miR-200c, miR-206, miR-1, miR-205, miR ebv-BHRF1-1, miR ebv-BHRF1-2, miR ebv-BHRF12-1, miR kshv-K3, miR kshv-K4-3p, miR kshv-mir-K2, miR kshv-mir K5, miR kshv-mir-K6-3p, miR kshv-mir-K7, miR kshv-mir-K11, miR-31, miR-196, miR-215, miR-155, miR-142-5p, miR-142-3p, miR-143, Hsa-mir-146a, Hsa-mir-146b, mCMV-miR-01-1, mCMV-miR-01-2, mCMV-miR-23-1, mCMV-miR-23-2, mCMV-miR-44-1, miR-133, miR-133b, miR-124, miR-126, miR-126-3p, miR-126-5p, miR-21, miR-22, miR-33) or pre-miRNA activity in a cell, e.g., a cell of a subject. The method includes contacting the cell with an effective amount of an oligonucleotide agent described herein. Such methods can be performed on a mammalian subject by administering to a subject one of the oligonucleotide agents/pharmaceutical compositions described herein.

In another aspect the invention features a method of decreasing levels of an RNA or protein that are encoded by a gene whose expression is down-regulated by an miRNA, e.g., an endogenous miRNA, such as miR-122, miR-16, miR-192, mir-194, miR-141, mRR-143, miR-181, miR-181a, miR-181c, miR-200c, miR-206, miR-1, miR-205, miR ebv-BHRF1-1, miR ebv-BHRF1-2, miR ebv-BHRF12-1, miR kshv-K3, miR kshv-K4-3p, miR kshv-mir-K2, miR kshv-mir-K5, miR kshv-mir-K6-3p, miR kshv-mir-K7, miR kshv-mir-K11, miR-31, miR-196, miR-215, miR-155, miR-142-5p, miR-142-3p, miR-143, Hsa mir 146a, Hsa-mir-146b, mCMV-miR-01-1, mCMV-miR-01-2, mCMV-miR-23-1, mCMV-miR-23-2, mCMV-miR-44-1, miR-133, miR-133b, miR-124, miR-126, miR-126-3p, miR-126-5p, miR-21, miR-22, or miR-33. The method includes contacting the cell with an effective amount of an oligonucleotide agent described herein, which includes a sequence that is substantially identical to the nucleotide sequence of the miRNA that binds to and effectively inhibits translation of the RNA transcribed from the gene. For example, the invention features a method of decreasing aldolase A protein levels in a cell. Similarly, the invention features a method of decreasing Ndrg3, Iqgap1, Hmgcr, and/or citrate synthase protein levels in a cell. The methods include contacting the cell with an effective amount of an oligonucleotide agent described herein (e.g., an oligonucleotide agent in Table 2), which is includes a sequence that is substantially identical to the nucleotide sequence of miR-122 (see Table 1).

In another aspect, the invention provides methods of decreasing expression of a target gene by providing an oligonucleotide agent to which a lipophilic moiety is conjugated, e.g., a lipophilic conjugated oligonucleotide agent described herein, to a cell. The oligonucleotide agent is preferably substantially identical to an miRNA (e.g., miR-122, miR-16, miR-192, miR-194, miR-141, mRR-143, miR-181, miR-181a, miR-181c, miR-200c, miR-206, miR-1, miR-205, miR ebv-BHRF1-1, miR ebv-BHRF1-2, miR ebv-BHRF12-1, miR kshv-K3, miR kshv-K4-3p, miR kshv-mir-K2, miR kshv-mir-K5, miR kshv-mir-K6-3p, miR kshv-mir-K7, miR kshv-mir-K11, miR-31, miR-196, miR-215, miR-155, miR-142-5p, miR-142-3p, miR-143, Hsa-mir-146a, Hsa-mir-146b, mCMV-miR-01-1, mCMV-miR-01-2, mCMV-miR-23-1, mCMV-miR-23-2, mCMV-miR-44-1, miR-133, miR-133b, miR-124, miR-126, miR-126-3p, miR-126-5p, miR-21, miR-22, or miR-33) or a pre-miRNA. In a preferred embodiment the conjugated oligonucleotide agent can be used to decrease expression of a target gene in an organism, e.g., a mammal, e.g., a human, or to decrease expression of a target gene in a cell line or in cells which are outside an organism. While not wishing to be bound by theory it is believed an mRNA transcribed from the target gene hybridizes to an endogenous miRNA, which consequently results in downregulation of mRNA expression. While not wishing to be bound by theory it is believed an mRNA transcribed from the target gene also hybridizes to the oligonucleotide agent featured in the invention consequently causes a decrease in mRNA expression that is greater than the decrease caused by the endogenous miRNA alone. In the case of a whole organism, the method can be used to decrease expression of a gene and treat a condition associated with a unwanted expression of the gene. For example, an oligonucleotide agent that targets miR-122 (e.g., an agent described herein) can be used to decrease expression of an aldolase A gene to treat a subject having, or at risk for developing, a disorder described herein, or any other disorder associated with aldolase A deficiency. Administration of an oligonucleotide agent that has a sequence substantially identical to miR-122 can also be used to decrease expression of an Ndrg3, Iqgap1, Hmgcr, or citrate synthase gene to treat a subject having, or at risk for developing, a disorder associated with a unwanted expression levels of any one of these genes.

### DESCRIPTION OF DRAWINGS

FIG. 5. Ligand conjugated oligonucleotide to modulate activity of miRNA: (a) ligand of interest is conjugated to the oligonucleotide via a tether and linker; (b) ligand of interest is conjugated to the oligonucleotide via a linker without a tether or tether without an additional linker and (c) a ligand of interest is attached directly to the oligonucleotide.

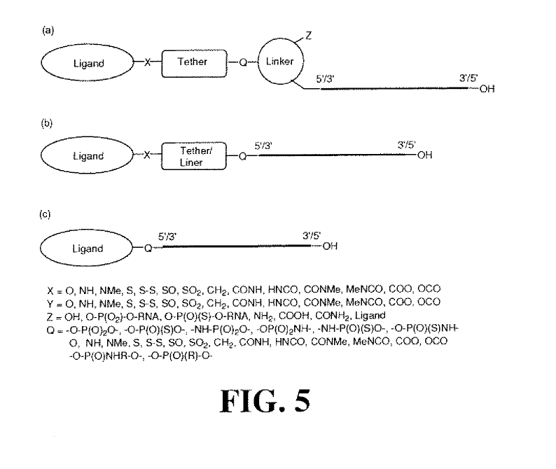

FIG. 6. Ligand conjugated double stranded oligonucleotide to modulate activity of miRNA: (a) ligand of interest is conjugated to the oligonucleotide via a tether and linker; (b) ligand of interest is conjugated to the oligonucleotide via a linker without a tether or tether without an additional linker and (c) a ligand of interest is attached directly to the oligonucleotide.

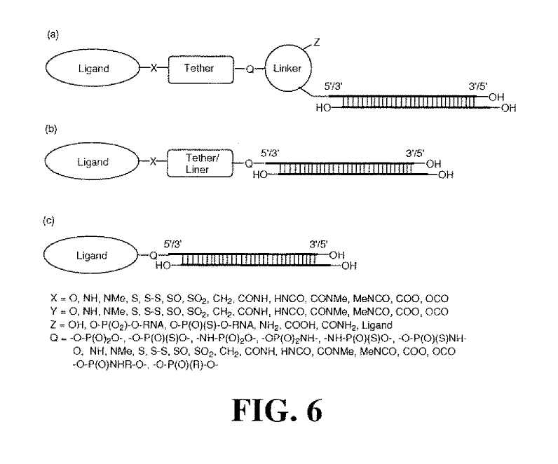

FIG. 7. Ligand conjugated antisense strand comprising partially double stranded oligonucleotides to modulate activity of miRNA. (a-c) ligand of interest is conjugated to the oligonucleotide via a tether and linker; (d-f) ligand of interest is conjugated to the oligonucleotide via a linker without a tether or tether without an additional linker and (g-i) a ligand of interest is attached directly to the oligonucleotide.

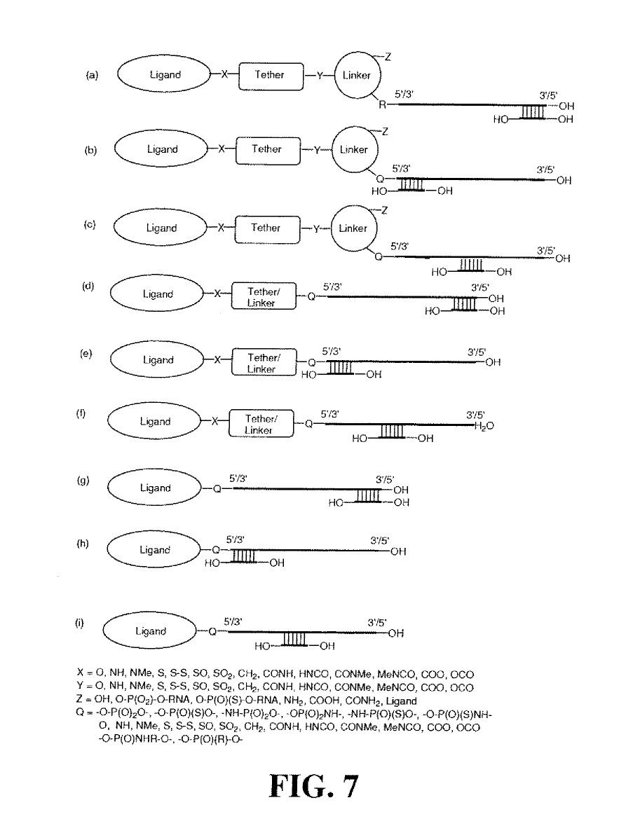

FIG. 8. Ligand conjugated partial sense strand comprising partially double stranded oligonucleotides to modulate activity of miRNA. (a-c) ligand of interest is conjugated to the oligonucleotide via a tether and linker; (d-f) ligand of interest is conjugated to the oligonucleotide via a linker without a tether or tether without an additional linker and (g-i) a ligand of interest is attached directly to the oligonucleotide.

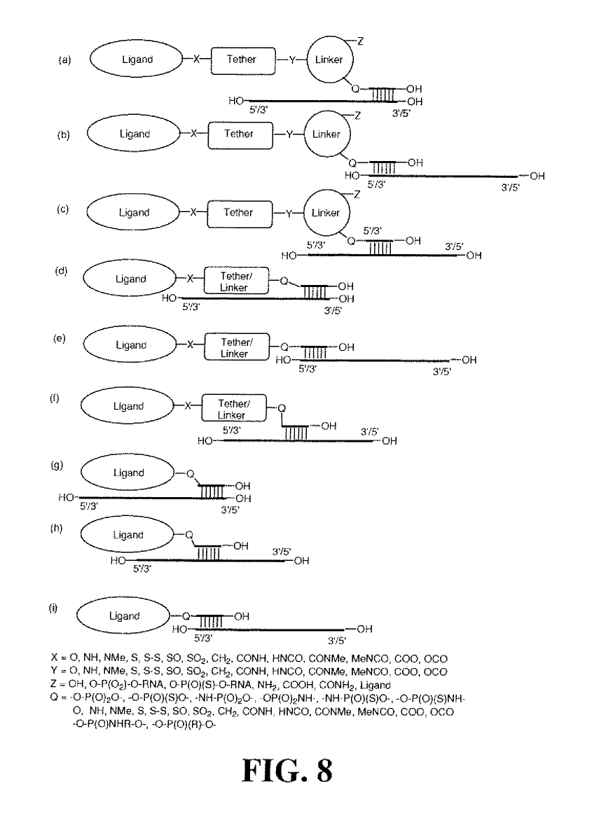

FIG. 9. Ligand conjugated partial hairpin oligonucleotides to modulate activity of miRNA. (a-b) ligand of interest is conjugated to either 3′ or 5′ end of the hairpin via a tether and linker; (c-d) ligand of interest is conjugated to the hairpin via a linker without a tether or tether without an additional linker and (e-f) a ligand of interest is attached directly to the oligonucleotide. The hairpin is comprised of nucleotides or non-nucleotide linkages.

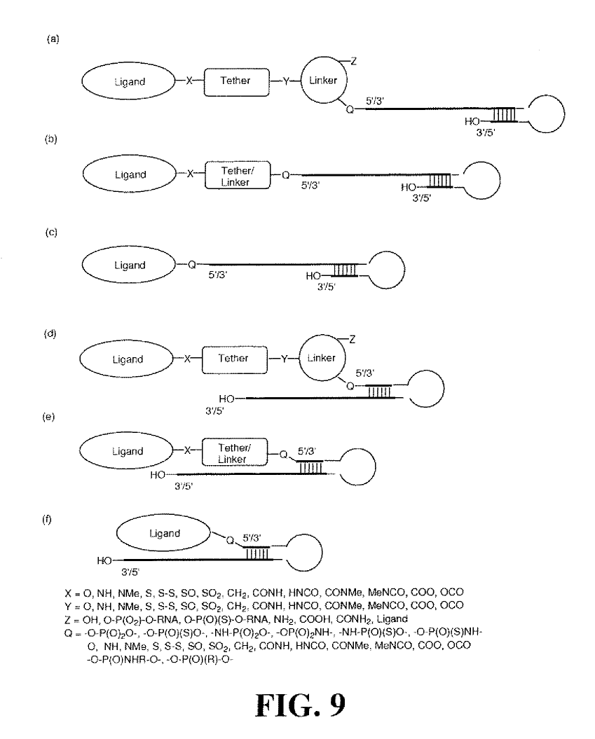

FIG. 10. Ligand conjugated hairpin oligonucleotides to modulate activity of miRNA. (a) ligand of interest is conjugated to either 3′ or 5′ end of the hairpin via a tether and linker; (b) ligand of interest is conjugated to the hairpin via a linker without a tether or tether without an additional linker and (c) a ligand of interest is attached directly to the oligonucleotide. The hairpin is comprised of nucleotides or non-nucleotide linkages.

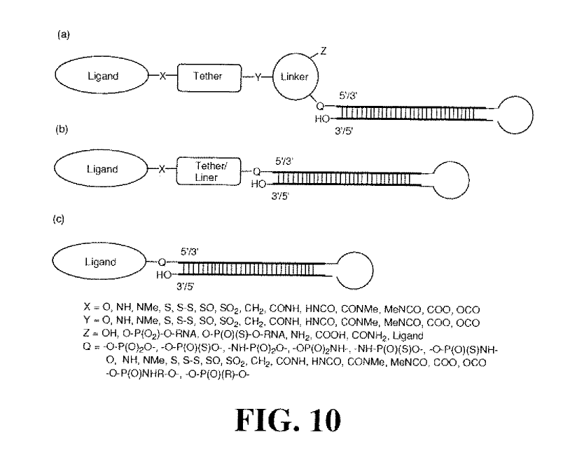

FIG. 11. Cholesterol conjugated oligonucleotides to modulate activity of miRNA. (a) 5′ cholesterol conjugate; (b) 3′ cholesterol conjugate and (c) cholesterol conjugate building blocks for oligonucleotide synthesis. The oligonucleotide can be miRNA, anti-miRNA, chemically modified RNA or DNA; DNA or DNA analogues for antisense application.

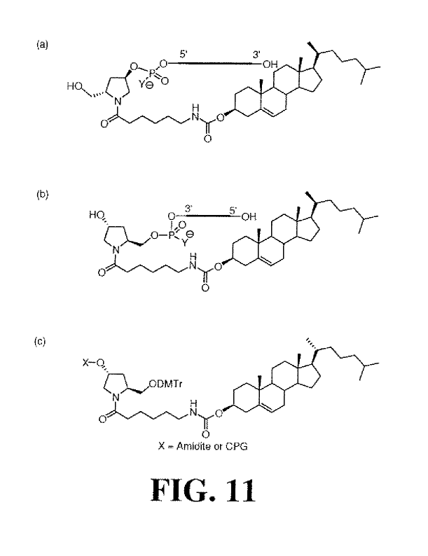

### DETAILED DESCRIPTION

The present invention is based in part on the discovery that activity levels of endogenous microRNAs (miRNAs) or pre-microRNAs (pre-miRNAs) can be augmented by an oligonucleotide agent including a sequence that is substantially identical to an endogenous miRNA that is administered, e.g., through systemic administration of the oligonucleotide agent, as well as by parenteral administration of such agents. Based on these findings, the present invention provides specific compositions and methods that are useful in enhancing the effect of miRNA and pre-miRNA activity levels, in e.g., a mammal, such as a human. In particular, the present invention provides specific compositions and methods that are useful for decreasing expression levels of an miRNA, e.g., miR-122, miR-16, miR-192, or miR-194, herein defined as supermirs.

In one aspect, the invention features supermirs. A supermir is a single-stranded, double stranded, partially double stranded or hairpin structured chemically modified oligonucleotide agents that consisting of, consisting essentially of or comprising at least 12 or more contiguous nucleotides substantially identical to an endogenous miRNA and more particularly, agents that include 12 or more contiguous nucleotides substantially complementary to a target sequence of an miRNA or pre-miRNA nucleotide sequence. As used herein partially double stranded refers to double stranded structures that contain less nucleotides than the complementary strand. In general, such partial double stranded agents will have less than 75% double stranded structure, preferably less than 50%, and more preferably less than 25%, 20% or 15% double stranded structure. FIGS. 1-3 provide representative structures of agents.

Preferably, a supermir featured in the invention includes a nucleotide sequence sufficiently complementary to hybridize to an miRNA target sequence of about 12 to 25 nucleotides, preferably about 15 to 23 nucleotides. More preferably, the target sequence differs by no more than 1, 2, or 3 nucleotides from a sequence shown in Table 1, and in one embodiment, the supermir is an agent shown in Table 2. In one embodiment, the supermir includes a non-nucleotide moiety, e.g., a cholesterol moiety. The non-nucleotide moiety can be attached, e.g., to the 3′ or 5′ end of the oligonucleotide agent. In a preferred embodiment, a cholesterol moiety is attached to the 3′ end of the oligonucleotide agent.

In some embodiments, the oligonucleotide agent is modified, for example, to further stabilize against nucleolytic degradation. Exemplary modifications include a nucleotide base or modification of a sugar moiety. The oligonucleotide agent can include modified linker agent such as a phosphorothioate in at least the first, second, or third internucleotide linkage at the 5′ or 3′ end of the nucleotide sequence. In one embodiment, the oligonucleotide agent includes a 2′-modified nucleotide, e.g., a 2′-deoxy, 2′-deoxy-2′-fluoro, 2′-O-methyl, 2′43-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In a particularly preferred embodiment, the oligonucleotide agent includes at least one 2′-O-methyl-modified nucleotide, and in some embodiments, all of the nucleotides of the oligonucleotide agent include a 2′-O-methyl modification. In some embodiments, the sugar moiety of the nucleotide can be replaced, for example, with a non-sugar moiety such as a PNA.

The oligonucleotide agent can be further modified so as to be attached to a ligand. The ligand can be selected, for example, to improve stability, distribution or cellular uptake of the agent, e.g., cholesterol or folate.

The oligonucleotide agent can further be in isolated form or can be part of a pharmaceutical composition used for the methods described herein, particularly as a pharmaceutical composition formulated for parental administration. The pharmaceutical compositions can contain one or more oligonucleotide agents, and in some embodiments, will contain two or more oligonucleotide agents, each one directed to a different miRNA.

A supermir that is substantially identical to a nucleotide sequence of an miRNA can be delivered to a cell or a human to aument the activity level of an endogenous miRNA, such as when insufficient miRNA activity, or unwanted activity of a target mRNA that hybridizes to the endogenous miRNA, is linked to a disease or disorder. In one embodiment, an supermir featured in the invention has a nucleotide sequence that is substantially identical to miR-122 (see Table 1), which hybridizes to numerous RNAs, including aldolase A mRNA, N-myc downstram regulated gene (Ndrg3) mRNA, IQ motif containing GTPase activating protein-1 (Iqgap1) mRNA, HMG-CoA-reductase (Hmgcr) mRNA, and citrate synthase mRNA and others. In a preferred embodiment, the supermir that is substantially identical to miR-122 is one of the sequences described herein (Table 2). Unwanted Aldolase A activity has been found to be associated with a variety of disorders, including hemolytic anemia, arthrogryposis complex congenita, pituitary ectopia, rhabdomyolysis, hyperkalemia. Thus a human who has or who is diagnosed as having any of these disorders or symptoms is a candidate to receive treatment with a supermir, such as a single-stranded oligonucleotide agent, that has a sequence substantially idencital to miR-122.

In some embodiments, a supermir featured in the invention has a nucleotide sequence that is substantially identical to an miRNA, e.g., miR-16, miR-192, or miR-194 (see Table 1).

In one embodiment, the supermir is selected from those shown in Table 2. The single-stranded oligonucleotide agents of Table 2 are have a nucleotide sequence substantially identical to mir-122 to and hybridize to the target sequence of mir-122.

TABLE 1
Exemplary miRNAs identified in mus musculus

| miRNA | Sequence | SEQ ID NO: |
|---|---|---|
| miR-122 | 5′-UGGAGUGUGACAAUGGUGUUUGU-3′ | 1 |
| miR-16 | 5′-UAGCAGCACGUAAAUAUUGGCG-3′ | 2 |
| miR-192 | 5′-CUGACCUAUGAAUUGACAGCC-3′ | 3 |
| miR-194 | 5′-UGUAACAGCAACUCCAUGUGGA-3′ | 4 |

TABLE 2
Oligo

| | | | | | |
|---|---|---|---|---|---|
| | | | Avail- | | |
| | | | ability | Cal. | Obs. |
| ID | Sequence | Modification | (mg) | mass | mass |

I. mir-122 and “Super Mirs”

| | | | | |
|---|---|---|---|---|
| 3035 | UGGAGUGUGACAAUGGUGUUUGU | Unmodified | 35 | 7422.3865 |

Mismatches

| | | | | |
|---|---|---|---|---|
| 3036 | UGGAAUGUGACAGUGUUGUGUGU | Complementary | 2 | 7422.3865 |
| | | to 3034 | | |
| | | (which has | | |
| | | 4 mm) | | |
| 3027 | usgsgsasasusgsusgsascsasgsusgsususgsusgsusgsusQ11 | Cholesterol, | | 8819.4235 |
| | | all 2′-OMe, | | |
| | | all PS, 4 mis- | | |
| | | matches | | |
| | | g to a, a to | | |
| | | g, g to u, | | |
| | | u to g | | |
| 3028 | usgsgaaugugacaguguugugusgsusQ11 | Cholesterol, all | | 8530.2427 |
| | | 2′-OMe, 2 + 4 | | |
| | | PS, 4 | | |
| | | mismatches | | |
| | | g to a, | | |
| | | a to g, | | |
| | | g to u, | | |
| | | u to g | | |

2′-F modifications

| | | | |
|---|---|---|---|
| 3023 | UfsGfsGfsAfsGfsUfsGfsUfsGfsAfsCfsAfsAfsUfsGfsGfsUfs | Cholesterol, | 8542.607 |
| | GfsUfsUfsUfsGfsUfsQ11 | all 2′-F, | |
| | | all PS | |
| 3024 | UfsGfsGfAfGfUfGfUfGfAfCfafAfUfGfGfUfGfUfUfsUfsGfsUf | Cholesterol, | 8269.4918 |
| | sQ11 | all 2′-F, | |
| | | all PS, | |
| | | 2 + 4 PS | |
| 3029 | UfsGfsGfsAfsAfsUfsGfsUfsGfsAfsCfsAfsGfsUfsGfsUfsUfs | Cholesterol, | 8542.607 |
| | GfsUfsGfsUfsGfsUfsQ11 | all 2′-F, | |
| | | all PS, 4 mis- | |
| | | matches | |
| | | g to a, | |
| | | a to g, | |
| | | g to u, | |
| | | u to g | |
| 3030 | UfsGfsGfAfAfUfGfUfGfAfCfAfGfUfGfUfUfGfUfGfsUfsGfsUf | Cholesterol, | 8269.4918 |
| | sQ11 | all 2′-F, | |
| | | 2 + 4 PS, 4 | |
| | | 4 mismatches | |
| | | g to a, | |
| | | a to g, | |
| | | g to u, | |
| | | u to g | |

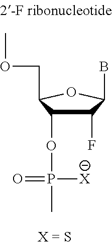

2′-OMOE modifications

| | | | |
|---|---|---|---|
| 3025 | TsGsGsAsGsTsGsTsGsAs^{m5}CsAsAsTsGsGsTsGsTsTsTs | Cholesterol, all | 9972.8984 |
| | GsTsQ11 | 2′-methoxy- | |
| | | ethyl, | |
| | | all PS | |
| 3026 | TsGsGAGTGTGA(m5C)AATGGTGTTsTsGsTsQ11 | Cholesterol, all | 9699.7832 |
| | | 2′-methoxy- | |
| | | ethyl, 2 + 4 PS | |
| 3031 | TsGsGsAsAsTsGsTsGsAs^{m5}CsAsGsTsGsTsTsTsGsTsGsTs | Cholesterol, all | 9972.8984 |
| | GsTsQ11 | 2′-methoxy- | |
| | | ethyl, all PS, | |
| | | 4 mismatches | |
| | | g to a, | |
| | | a to g, | |
| | | g to u, | |
| | | u to g | |
| 3032 | TsGsGAATFTFA^{m5}CAGTGTTGTGsTsGsTsQ11 | Cholesterol, all | 9699.7832 |
| | | 2′-methoxy- | |
| | | ethyl, | |
| | | 2 + 4 PS, | |
| | | 4 mismatches | |
| | | g to a, | |
| | | a to g, | |
| | | g to u, | |
| | | u to g | |

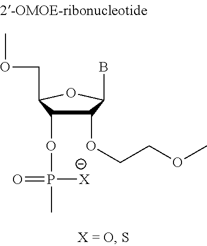

Purine modification

| | | | |
|---|---|---|---|
| 3344 | UGGIGUGUGICIIUG GUGUUUG | All ribo, | 7120.1597 |
| | | all Adenosines | |
| | | replaced with | |
| | | inosine | |

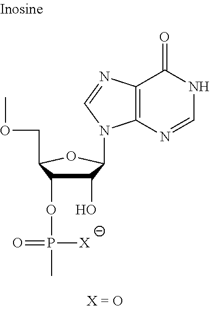

P = S modifications

| | | | | |
|---|---|---|---|---|
| 3544 | UsGsGAGUGUGACAAUGGUGUUUsGsU | Parent 3035, | 60 | 7486.6489 |
| | | 2 × PS each | | |
| | | end | | |

Cholesterol modifications

| | | | | |
|---|---|---|---|---|
| 3627 | UGGAGUGUGACAAUGGUGUUUGsUsL10 | Parent 3035, | 115 | 8159.4341 |
| | | cholesterol, | | |
| | | 2 PS on 3′ end | | |
| 3224 | usgsgaaggugacaguguuguususgsugL10 | Cholesterol, all | 115 | 8546.3083 |
| | | 2′-OMe, , | | |
| | | 2 + 4 PS | | |
| 3629 | uGGAGuGuGAcAAuGGuGuuuGsusL10 | Parent 3035, | 70 | 8299.7001 |
| | | all Py | | |
| | | 2′-OMe, 2 PS, | | |
| | | 3′-cholesterol | | |
| 3021 | usgsgsasgsusgsusgsascsasasusgsgsusgsusususgsusQ11 | Cholesterol, all | | 8819.4235 |
| | | 2′-OMe, all PS | | |
| 3022 | usgsgagugugacaaugguguususgsusQ11 | Cholesterol, all | | 8546.3083 |
| | | 2′-OMe, all PS | | |

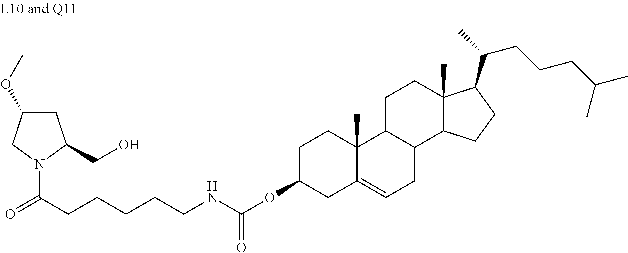

2′-OMe modifications

| | | | | |
|---|---|---|---|---|
| 3545 | UsGsGAGUGUGACAauGGUGUUUsGsU | Parent 3035, | 60 | 7514.7021 |
| | | 2 × PS | | |
| | | each and, | | |
| | | 2 × 2′-OMe | | |
| 3546 | UGGAGUGUGACAauGGUGUUUGU | Parent 3035, | | 7450.4397 |
| | | 2 × 2′-OMe | | |

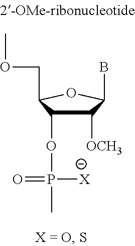

2′-OMe and PS modifications

| | | | | |
|---|---|---|---|---|
| 3547 | UsGsGAGUGUGACAasusGGUGUUUsGsU | Parent 3035, | 29 | 7546.8333 |
| | | 2 × 2′-OMe & | | |
| | | PS, 2 × PS | | |
| | | each end | | |
| 3628 | uGGAGuGuGAcAAuGGuGuuusGsu | Parent 3035, | 16 | 7594.7837 |
| | | All Py | | |
| | | 2′-OMe, 2 PS | | |
| 3849 | usgsgagugugacaauggugusususgsu | Parent 3035, | 64 | 7841.3919 |
| | | all 2′-OMe, | | |
| | | 2 + 4 PS | | |

5-(aminoethyl-3-acrylimido) thymidine

| | | | |
|---|---|---|---|
| 30055 | UGGAGUGUGACAAY13GGUGUUUGU | U displaced with | 7518.5169 |
| | | 5-(aminoethyl- | |
| | | 3-acrylimido) | |
| | | thymidine | |
| 30056 | UGGAGUGY13GACAAUGGUGUUUGU | U displaced with | 7518.5169 |
| | | 5-(aminoethyl- | |
| | | 3-acrylimido) | |
| | | thymidine | |
| 30057 | UGGAGUGY13GACAAY13GGUGUUUGU | 2 × U displaced | 7614.6473 |
| | | with 5-(amino- | |
| | | ethyl-3-acylimido) | |
| | | thymidine | |

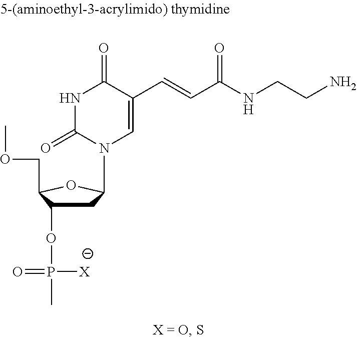

Biotin and 5-(aminoethyl-3-acrylimido) thymidine Conjugates

| | | | |
|---|---|---|---|
| 30058 | UGGAGUGUGACAAY13GGUGUUUGUL29 | L29 = | 8234.398 |
| | | N-(biotinyl- | |
| | | aminododecyl- | |
| | | carbox- | |
| | | amidocaproyl)-4- | |
| | | hydroxyprolinol | |
| | | Y13 = with | |
| | | 5-(aminoethyl-3- | |
| 30059 | UGGAGUGY13GACAAUGGUGUUUGUL29 | | 8234.398 |
| 30099 | UGGAGUGY13GACAAY13GGUGUUUGUL29 | | 8330.5284 |
| 30104 | UsGGAGUGUGACAAY13GGUGUUUGUsL29 | | 8266.5292 |
| 30105 | UsGGAGUGY13GACAAUGGUGUUUGUsL29 | | 8266.5292 |
| 30106 | UsGGAGUGY13GACAAY13GGUGUUUGUsL29 | 2 × Y13 and | 8362.6596 |
| | | biotin, 2 PS | |

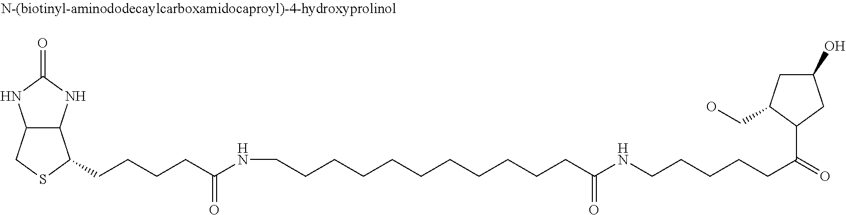

5-(psoralencarboxamidoethyl-3-acrylimido) thymidine

| | | | |
|---|---|---|---|
| 30067 | UGGAGUGUGACAAY13GGUGUUUGU | Y14 = | 7788.7538 |
| | | 5-(psoralencarbox- | |
| | | amidoethyl- | |
| | | 3-acrylimido) | |
| | | thymidine-3′- | |
| | | phosphate | |
| 30068 | UGGAGUGY14GACAAUGGUGUUUGU | | 7788.7538 |
| 30069 | UGGAGUGY14GACAAY14GGUGUUUGU | 2 × Y14 | 8155.1211 |

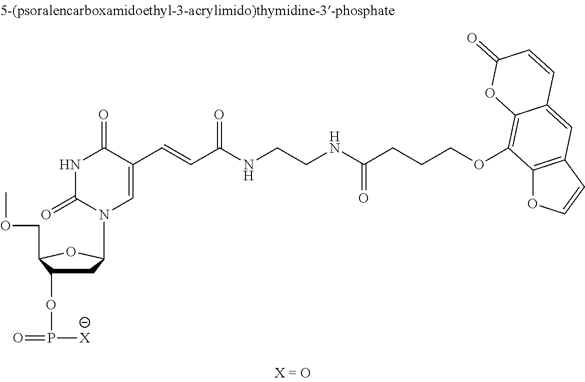

Psoralene and Biotin

| | | | |
|---|---|---|---|
| 30070 | UGGAGUGUGACAAY14GGUGUUUGUL29 | Y14 = | 8504.6349 |
| | | 5-(psoralencarbox- | |
| | | amidoethyl-3- | |
| | | acrylimido) | |
| | | thymidine-3′- | |
| | | phosphate | |
| | | L29 = | |
| | | N-(biotinyl- | |
| | | aminododecyl- | |
| | | carboxamido- | |
| | | caproyl)-4- | |
| | | hydroxyprolinol | |
| 30071 | UGGAGUGY14GACAAY14GGUGUUUGUL29 | 2 × Y14 | 8871.0022 |
| | | and biotin | |
| 30111 | UsGGAGUGUGACAAY14GGUGUUUGUsL29 | Y14, biotin, | 8536.7661 |
| | | 2 PS | |
| 30112 | UsGGAGUGY14GACAAUGGUGUUUGUsL29 | Y14, biotin, | 8536.7661 |
| | | 2 PS | |
| 30113 | UsGGAGUGY14GACAAY14GGUGUUUGUsL29 | 2 × Y14, biotin, | 8903.1334 |
| | | 2 PS | |
| 30121 | UGGAGUGY14GACAAUGGUGUUUGUL29 | Y14, biotin, | 8504.6349 |
| | | No PS | |

In one aspect, the invention features a supermir, such as a single-stranded oligonucleotide agent, that includes a nucleotide sequence that is substantially identical to a nucleotide sequence of an miRNA, such as an endogenous miRNA listed in Table 1. An oligonucleotide sequence that is substantially identical to an endogenous miRNA sequence is 70%, 80%, 90%, or more identical to the endogenous miRNA sequence. Preferably, the agent is identical in sequence with an endogenous miRNA. A supermir, e.g., one that is substantially identical to a nucleotide sequence of an miRNA, can be delivered to a cell or a human to replace or supplement the activity of an endogenous miRNA, such as when an miRNA deficiency is linked to a disease or disorder, or aberrant or unwanted expression of the mRNA that is the target of the endogenous miRNA is linked to a disease or disorder. In one embodiment, a supermir featured in the invention can have a nucleotide sequence that is substantially identical to miR-122 (see Table 1). An miR-122 binds to numerous RNAs including aldolase A mRNA, which has been shown to be overexpressed in different cancers, including lung cancer and breast cancer, and is overexpressed in adenocarcinomas of various different tissues origins. Thus a single stranded supermir that is substantially identical to miR-122 can be administered as a therapeutic composition to a subject having or at risk for developing lung cancer or breast cancer, for example.

An miR-122 binds other mRNAs, including N-myc downstram regulated gene (Ndrg3) mRNA, IQ motif containing GTPase activating protein-1 (Iqgap1) mRNA, HMG-CoA-reductase (Hmgcr) mRNA, and citrate synthase mRNA. Iqgap1 overexpression is associated with gastric cancer and colorectal cancer. Thus a single stranded supermir that is substantially identical to miR-122 can be useful for downregulating Iqgap1 expression, and can be administered as a therapeutic composition to a subject having or at risk for developing gastric cancer and colorectal cancer. Hmgcr inhibitors are useful to treat hyperglycemia and to reduce the risk of stroke and bone fractures. Thus a single stranded supermir that is substantially identical to miR-122 can be useful for downregulating Hmgcr expression, and can be administered as a therapeutic composition to a subject having or at risk for developing hyperglycemia, stroke, or a bone fracture. A single stranded supermir that is substantially identical to miR-122 can be administered as a therapeutic composition to a subject having or at risk for developing a disorder characterized by the aberrant or unwanted expression of any of these genes, or any other gene downregulated by miR-122.

In one embodiment, a supermir, such as a single-stranded oligonucleotide agent, can have a nucleotide sequence that is substantially identical to, e.g., miR-16, miR-192, or miR-194. Single-stranded oligonucleotide agents that are substantially identical to at least a portion of an miRNA, such as those described above, can be administered to a subject to treat the disease or disorder associated with the downregulation of an endogenous miRNA, or the aberrant or unwanted expression of an mRNA target of the endogenous miRNA.

In one aspect, the invention features a method of supplementing the effect of an miRNA or pre-miRNA in a cell of a subject, e.g., a human subject. The method includes the step of administering a supermir to the subject, where the supermir is substantially single-stranded and includes a sequence that is substantially complementary to 12 to 23 contiguous nucleotides, and preferably 15 to 23 contiguous nucleotides, of a target sequence of an miRNA or pre-miRNA nucleotide sequence. Preferably, the target sequence differs by no more than 1, 2, or 3 nucleotides from a microRNA or pre-microRNA sequence, such as a microRNA sequence shown in Table 1.

In one embodiment, the methods featured in the invention are useful for reducing the level of an mRNA that is the target of an endogenous miRNA (e.g., miR-122, miR-16, miR-192 or miR-194) or pre-miRNA in a cell, e.g, in a cell of a subject, such as a human subject. Such methods include contacting the cell with a supermir, such as a single-stranded oligonucleotide agent, described herein for a time sufficient to allow uptake of the supermir into the cell.

In another aspect, the invention features a method of making a supermir, such as a single-stranded oligonucleotide agent, described herein. In one embodiment, the method includes synthesizing an oligonucleotide agent, including incorporating a nucleotide modification that stabilizes the supermir against nucleolytic degradation.

In another aspect, the invention features a pharmaceutical composition including a supermir, such as a single-stranded oligonucleotide agent, described herein, and a pharmaceutically acceptable carrier. In a preferred embodiment, the supermir, such as a single-stranded oligonucleotide agent, included in the pharmaceutical composition hybridizes to an mRNA target of, e.g., miR-122, miR-16, miR-192, or miR-194.

In another aspect, the invention features a method of supplementing miRNA activity levels (e.g., miR-122, miR-16, miR-192, or miR-194 expression) or pre-miRNA expression in a cell, e.g., a cell of a subject. The method includes contacting the cell with an effective amount of a supermir, such as a single-stranded oligonucleotide agent, described herein, which is substantially complementary to the nucleotide sequence of the target miRNA or the target pre-miRNA. Such methods can be performed on a mammalian subject by administering to a subject one of the oligonucleotide agents/pharmaceutical compositions described herein.

In another aspect, the invention features a method of decreasing levels of an RNA or protein that is encoded by a gene whose expression is down-regulated by an miRNA, e.g., an endogenous miRNA, such as miR-122, miR-16, miR-192 or mir-194. The method includes contacting the cell with an effective amount of a supermir, such as a single-stranded oligonucleotide agent, described herein, which is substantially identical to the nucleotide sequence of the miRNA that binds to and effectively inhibits translation of the RNA transcribed from the gene. For example, the invention features a method of decreasing aldolase A protein levels in a cell. Similarly, the invention features a method of decreasing Ndrg3, Iqgap1, Hmgcr, and/or citrate synthase protein levels in a cell. The methods include contacting the cell with an effective amount of a supermir described herein (e.g., described in Table 2), which is substantially identical to the nucleotide sequence of miR-122 (see Table 1).

Preferably, a supermir, such as a single-stranded oligonucleotide agent, (a term which is defined below) will include a ligand that is selected to improve stability, distribution or cellular uptake of the agent. Compositions featured in the invention can include conjugated single-stranded oligonucleotide agents as well as conjugated monomers that are the components of or can be used to make the conjugated oligonucleotide agents. The conjugated oligonucleotide agents can modify gene expression by targeting and binding to a nucleic acid, such as the target mRNA of an miRNA (e.g., miR-122, miR-16, miR-192, or miR-194) or pre-miRNA.

In a preferred embodiment, the ligand is a lipophilic moiety, e.g., cholesterol, which enhances entry of the antagomir, such as a single-stranded oligonucleotide agent, into a cell, such as a hepatocyte, synoviocyte, myocyte, keratinocyte, leukocyte, endothelial cell (e.g., a kidney cell), B-cell, T-cell, epithelial cell, mesodermal cell, myeloid cell, neural cell, neoplastic cell, mast cell, or fibroblast cell. In some embodiments, a myocyte is a smooth muscle cell or a cardiac myocyte. A fibroblast cell can be a dermal fibroblast, and a leukocyte can be a monocyte. In another embodiment, the cell is from an adherent tumor cell line derived from a tissue, such as bladder, lung, breast, cervix, colon, pancreas, prostate, kidney, liver, skin, or nervous system (e.g., central nervous system). In some preferred embodiments, the ligand is a folate ligand.

In another aspect, the invention provides methods of decreasing expression of a target gene by providing a supermir to which a lipophilic moiety is conjugated, e.g., a lipophilic conjugated supermir described herein, to a cell. The supermir preferably hybridizes to an miRNA (e.g., miR-122, miR-16, miR-192, or miR-194) or a pre-miRNA. In a preferred embodiment the conjugated supermir can be used to increase expression of a target gene in an organism, e.g., a mammal, e.g., a human, or to increase expression of a target gene in a cell line or in cells which are outside an organism. An mRNA transcribed from the target gene hybridizes to an endogenous miRNA, which consequently results in downregulation of mRNA expression. A supermir, such as a single-stranded oligonucleotide agent, featured in the invention hybridizes to the target mRNA of the endogenous miRNA and consequently causes a further decrease in mRNA expression. In the case of a whole organism, the method can be used to decrease expression of a gene and treat a condition associated with a aberrant or unwanted target gene expression. For example, a supermir, such as a single-stranded oligonucleotide agent, that targets the same mRNA sequence as miR-122 can be used to decrease expression of an aldolase A gene to treat a subject having, or at risk for developing, cancer, or any other disorder associated with aldolase A overexpression. Administration of a supermir, such as a single-stranded oligonucleotide agent, that targets the same mRNA sequence as miR-122 can also be used to decrease expression of an Ndrg3, Iqgap1, Hmgcr, or citrate synthase gene to treat a subject having, or at risk for developing, a disorder associated with a aberrant or unwanted expression of any one of these genes.

In one embodiment, the supermir, such as a single-stranded oligonucleotide agent, to which a lipophilic moiety is conjugated is used to decrease expression of a gene in a cell that is not part of a whole organism, such as when the cell is part of a primary cell line, secondary cell line, tumor cell line, or transformed or immortalized cell line. Cells that are not part of a whole organism can be used in an initial screen to determine if a supermir, such as a single-stranded oligonucleotide agent, is effective in decreasing target gene expression levels. A test in cells that are not part of a whole organism can be followed by test of the supermir in a whole animal. In some embodiments, the supermir that is conjugated to a lipophilic moiety is administered to an organism, or contacted with a cell that is not part of an organism, in the absence of (or in a reduced amount of) other reagents that facilitate or enhance delivery, e.g., a compound which enhances transit through the cell membrane. (A reduced amount can be an amount of such reagent which is reduced in comparison to what would be needed to get an equal amount of nonconjugated agent into the target cell). For example, the supermir that is conjugated to a lipophilic moiety is administered to an organism, or contacted with a cell that is not part of an organism, in the absence (or reduced amount) of (i) an additional lipophilic moiety; (ii) a transfection agent (e.g., an ion or other substance which substantially alters cell permeability to an oligonucleotide agent); or (iii) a commercial transfecting agent such as Lipofectamine™ (Invitrogen, Carlsbad, Calif.), Lipofectamine 2000™, TransIT-TKO™ (Minis, Madison. WI), FuGENE 6 (Roche, Indianapolis, Ind.), polyethylenimine, X-tremeGENE Q2 (Roche, Indianapolis, Ind.), DOTAP, DOSPER, Metafectene™ (Biontex, Munich, Germany), and the like.

Exemplary delivery vehicles for an oligonucleotide agent featured herein, include lipid (e.g., cationic lipid) containing vehicles (e.g., liposomes), viral containing vehicles (e.g., vectors), polymer containing vehicles (e.g., biodegradable polymers or dendrimers), and peptide containing vehicles (e.g., a penetration peptide), exosomes, and bacterially-derived, intact minicells. In a preferred example the delivery vehicle includes more than one component. For example, it can include one or more lipid moieties, one or more peptides, one or more polymers, one or more viral vectors, or a combination thereof in a preferred embodiment, the delivery vehicle is an association complex such as a liposome. A liposome generally includes a plurality of components such as one or more of a cationic lipid (e.g., an amino lipid), a targeting moiety, a fusogenic lipid, a PEGylated lipid.

In some embodiments, the PEG-lipid is a targeted PEG-lipid. For example, a liposome can include a nucleic acid and an amine-lipid and a PEGylated lipid. In some embodiments, the PEG-lipid is a targeted PEG-lipid. In some embodiments, the preparation also includes a structural moiety such as cholesterol.

Exemplary Candidate Delivery Vehicles

An oligoneucleotide agent can be delivered using a variety of delivery vehicles including those containing one or more of the following: cationic lipids, cationic liposomes, neutral and zwitterionic lipids and liposomes, peptides (neutral, anionic and cationic; hydrophobic), dendrimers (neutral, anionic and cationic), polymers, emulsions, intralipids, omega-3 and related natural formulations, microemulsions and nanoemulsions, nanoparticles, nanosystems with targeting groups, nanosystems with endosomal releasing groups, polymeric micelles, polymeric vesicles, PEIs and polyamines, lipophilic polyamines, and hydrogels.

Exemplary delivery vehicles include peptide containing vehicles, collagen containing vehicles, viral vector containing vehicles, polymer containing vehicles, lipid containing (e.g., cationic lipids, PEG containing lipids, etc.). In some embodiments, the delivery vehicle includes a combination of one or more of the delivery components described above. Exemplary delivery vehicles, which can be evaluated using a screening model described herein include, but are not limited to the following: Exosomes such as those described in US 20070298118; bacterially-derived, intact minicells, for example, as described in US 20070298056; complexes including RNA and peptides such as those described in US 20070293657; cationic lipids, non-cationic lipids, and lipophilic delivery-enhancing compounds such as those described in US 20070293449); Carbohydrate-Derivatized Liposomes (e.g., as described in US 20070292494); siRNA-hydrophilic polymer conjugates (e.g., as described in US 20070287681); lipid and polypeptide based systems such as those described in US 20070281900; organic cation containing systems such as those described in US 20070276134 and US 20070213257; cationic peptide containing systems such as those described in US 20070275923; polypeptide containing systems such as those disclosed in US 20060040882; virus-phage particle containing systems such as those described in US 20070274908; elastin-like polymer containing systems such as those described in US 20070265197; non-immunogenic, hydrophilic/cationic block copolymers such as those described in US 20070259828; carrier linked conjugate containing systems such as those described in US 20070258993; biodegradable cationic polymer containing systems such as those described in US 20070243157; chemically modified polycation polymer containing systems such as those described in US 20070231392; collagen containing systems such as those described in US 20070218038; glycopolymer-based particle containing systems such as those described in US 20070202076; biologically active block copolymer containing systems such as those described in US 20070155907; nanoparticle containing systems such as those described in US 20070155658; amphoteric liposome containing systems such as those described in US 20070104775; lipid earlier containing systems such as those described in US 20070087045; electroporation systems such as those described in US 20070059832; macromer-melt formulations such as those described in US 20070053954; liposome containing systems such as those described in US 20070042031 and US 20050002999 and US 20050002998; lipid based formulations such as those described in US 20060008910; US 20050014962; US 20060240093, US 20050064595, and US 20060083780; polymer conjugate containing systems such as those described in US 20070041932 and US 20050008617; hydrophobic nanotube and nanoparticle containing systems such as those described in US 20060275371; functional synthetic molecule and macromolecule containing systems such as those described in US 20060241071; polymeric micelle containing systems such as those disclosed in US 20060240092; sugar-modified liposome containing systems such as those described in US 20060193906; cyclic amidinium-containing systems such as those described in US 20060039860 and US 20030220289; peptide containing compositions such as those described in US 20060035815 and US 20050239687; viral vector containing systems such as those described in US 20060009408, US 20030157691, and tRNA vector systems such as those described in US 20050203047; nanocell drug delivery systems such as those described in US 20050266067; polymerized formamide containing systems such as those described in US 20050265957; intranasal delivery systems such as those described in US 20050265927; nanoparticle systems such as those described in US 20050260276; biodegradable polymer-peptide containing systems such as those described in US 20050191746 and biodegradable polyacetal containing systems such as those described in US 20050080033; biodegradable cationic polymer containing systems such as those described in US 20060258751; biodegradable poly(beta-amino ester) containing systems such as those described in US 20040071654; polyethyleneglycol-modified lipid containing systems such as those described in US 20050175682; virally-encoded RNA systems such as those described in US 20050171041, US 20040023390, US 20030138407; adenoviral vector systems such as those described in US 20040161848 and US 20040096843; carrier complex containing systems such as those described in US 20050158373; compositions comprising amphipathic compounds and polycations such as those described in US 20050143332, US 20040137064, and US 20030125281; delivery peptide and dendrimer containing compositions such as those described in US 20040204377; polyampholyte containing compositions such as those described in US 20040162235; and microcapsule containing systems such as those described in US 20040115254 and formulations described in PCT/US2007/080331. Each of the references above is incorporated by reference herein in its entirety.

In some preferred embodiments one or more of the delivery vehicles can be formed into a particle such as a liposome or other association complex. The nucleic acid-based agent can be encapsulated or partially encapsulated in the particle delivery vehicle. In some embodiments, the nucleic acid-based agent is admixed with one or more delivery vehicles described herein.

In some embodiments, the nucleic acid-based agent is bound to a delivery vehicle described herein. For example, the nucleic acid-based agent can be bound to a delivery vehicle through hydrostatic interactions, ionic interactions, hydrogen bonding interactions or through a covalent bond.

In some embodiments, the nucleic acid-based agent is entrapped or entrained within a delivery vehicle.

Association Complexes

Association complexes can be used to administer a nucleic acid based therapy such as an oligonucleotide agent described herein. The association complexes disclosed herein can be useful for packaging an oligonucleotide.

Association complexes can include a plurality of components. In some embodiments, an association complex such as a liposome can include an oligonucleotide agent described herein, a cationic lipid such as an amino lipid. In some embodiments, the association complex can include a plurality of therapeutic agents, for example two or three single or double stranded nucleic acid moieties targeting more than one gene or different regions of the same gene. Other components can also be included in an association complex, including a PEG-lipid or a structural component, such as cholesterol. In some embodiments the association complex also includes a fusogenic lipid or component and/or a targeting molecule. In some preferred embodiments, the association complex is a liposome including an oligonucleotide agent such as dsRNA, a lipid, a PEG-lipid, and a structural component such as cholesterol.

In a preferred embodiment, the supermir is suitable for delivery to a cell in viva, e.g., to a cell in an organism. In another embodiment, the supermir is suitable for delivery to a cell in vitro, e.g., to a cell in a cell line.

A supermir to which a lipophilic moiety is attached can have a sequence substantially identical to any miRNA (e.g., miR-122, miR-16, miR-192, or miR-194) or pre-miRNA described herein and can be delivered to any cell type described herein, e.g., a cell type in an organism, tissue, or cell line. Delivery of the supermir can be in vivo, e.g., to a cell in an organism, or in vitro, e.g., to a cell in a cell line.

In another aspect, the invention provides compositions including single-stranded oligonucleotide agents described herein, and in particular, compositions including a supermir to which a lipophilic moiety is conjugated, e.g., a lipophilic conjugated supermir that hybridizes to miR-122, miR-16, miR-192, or miR-194. In a preferred embodiment the composition is a pharmaceutically acceptable composition.

In one embodiment the composition is suitable for delivery to a cell in vivo, e.g., to a cell in an organism. In another aspect, the supermir is suitable for delivery to a cell in vitro, e.g., to a cell in a cell line.

An “supermir” or “oligonucleotide agent” of the present invention referres to a single stranded, double stranded or partially double stranded oligomer or polymer of ribonucleic acid (RNA) or deoxyribonucleic acid (DNA) or both or modifications thereof, which has a nucleotide sequence that is substantially identical to an miRNA and that is antisense with respect to its target. This term includes oligonucleotides composed of naturally-occurring nucleobases, sugars and covalent internucleoside (backbone) linkages and which contain at least one non-naturally-occurring portion which functions similarly. Such modified or substituted oligonucleotides are preferred over native forms because of desirable properties such as, for example, enhanced cellular uptake, enhanced affinity for nucleic acid target and increased stability in the presence of nucleases. In a preferred embodiment, the supermir does not include a sense strand, and in another preferred embodiment, the supermir does not self-hybridize to a significant extent. An supermir featured in the invention can have secondary structure, but it is substantially single-stranded under physiological conditions. An supermir that is substantially single-stranded is single-stranded to the extent that less than about 50% (e.g., less than about 40%, 30%, 20%, 10%, or 5%) of the supermir is duplexed with itself. FIGS. 1-3 provide representative structures of supermirs.

The supermir can include a hairpin segment, e.g., sequence, preferably at the 3′ end can self hybridize and form a duplex region, e.g., a duplex region of at least 1, 2, 3, or 4 and preferably less than 8, 7, 6, or n nucleotides, e.g., 5 nucleotides. The duplexed region can be connected by a linker, e.g. a nucleotide linker, e.g., 3, 4, 5, or 6 dTs, e.g., modified dTs. In another embodiment the supermir is duplexed with a shorter oligo, e.g., of 5, 6, 7, 8, 9, or 10 nucleotides in length, e.g., at one or both of the 3′ and 5′ end or at one end and in the non-terminal or middle of the supermir.

“Substantially complementary” means that two sequences are substantially complementary that a duplex can be formed between them. The duplex may have one or more mismatches but the region of duplex formation is sufficient to down-regulate expression of the target nucleic acid. The region of substantial complementarity can be perfectly paired. In other embodiments, there will be nucleotide mismatches in the region of substantial complementarity. In a preferred embodiment, the region of substantial complementarity will have no more than 1, 2, 3, 4, or 5 mismatches.

The oligonucleotide agents featured in the invention include oligomers or polymers of ribonucleic acid (RNA) or deoxyribonucleic acid (DNA) or both or modifications thereof. This term includes oligonucleotides composed of naturally occurring nucleobases, sugars, and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for nucleic acid target, and/or increased stability in the presence of nucleases. The oligonucleotide agents can be about 12 to about 30 nucleotides long, e.g., about 15 to about 25, or about 18 to about 25 nucleotides long (e.g., about 19, 20, 21, 22, 23, 24 nucleotides long).

The oligonucleotide agents, e.g., supermirs, featured in the invention can target RNA, e.g., an target RNA sequence of an endogenous pre-miRNA or miRNA of the subject or an endogenous pre-miRNA or miRNA of a pathogen of the subject. For example, the oligonucleotide agents can target the mRNA sequence of an endogenous miRNA of the subject, such as miR-122, miR-16, miR-192, or miR-194. Such single-stranded oligonucleotides can be useful for the treatment of diseases involving biological processes that are regulated by miRNAs, including developmental timing, differentiation, apoptosis, cell proliferation, organ development, and metabolism.

### MicroRNA-Type Oligonucleotide Agents

The oligonucleotide agents featured in the invention include microRNA-type (miRNA-type) oligonucleotide agents, e.g., the miRNA-type oligonucleotide agents listed in Table 2. MicroRNAs are small noncoding RNA molecules that are capable of causing post-transcriptional silencing of specific genes in cells such as by the inhibition of translation or through degradation of the targeted mRNA. An miRNA can be completely complementary or can have a region of noncomplementarity with a target nucleic acid, consequently resulting in a “bulge” at the region of non-complementarity. The region of noncomplementarity (the bulge) can be flanked by regions of sufficient complementarity, preferably complete complementarity to allow duplex formation. Preferably, the regions of complementarity are at least 8, 9, or 10 nucleotides long. An miRNA can inhibit gene expression by repressing translation, such as when the microRNA is not completely complementary to the target nucleic acid, or by causing target RNA degradation, which is believed to occur only when the miRNA binds its target with perfect complementarity. The invention also can include double-stranded precursors of miRNAs that may or may not form a bulge when bound to their targets.

An miRNA or pre-miRNA can be 18-100 nucleotides in length, and more preferably from 18-80 nucleotides in length. Mature miRNAs can have a length of 19-30 nucleotides, preferably 21-25 nucleotides, particularly 21, 22, 23, 24, or 25 nucleotides. MicroRNA precursors typically have a length of about 70-100 nucleotides and have a hairpin conformation. MicroRNAs are generated in viva from pre-miRNAs by the enzymes Dicer and Drosha, which specifically process long pre-miRNA into functional miRNA. The miRNA-type oligonucleotide agents, or pre-miRNA-type oligonucleotide agents featured in the invention can be synthesized in viva by a cell-based system or in vitro by chemical synthesis. MicroRNA-type oligonucleotide agents can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting. Methods of synthesis and chemical modifications are described in greater detail below.

Given a sense strand sequence (e.g., the sequence of a sense strand of a cDNA molecule), an miRNA-type oligonucleotide agent featured in the invention, e.g., a supermir, can be designed according to the rules of Watson and Crick base pairing. The miRNA-type supermir can be complementary to a portion of an RNA, e.g., an mRNA. For example, the miRNA-type oligonucleotide agent can be substantially identical to an miRNA endogenous to a cell, such as miR-122, miR-16, miR-192, or miR-194. An miRNA-type oligonucleotid agent can be, for example, from about 12 to 30 nucleotides in length, preferably about 15 to 28 nucleotides in length (e.g., 16, 17, 18, 19, 20, 21, 22, 23, 24, 25, 26, or 27 nucleotides in length).

In particular, an miRNA-type oligonucleotide agent featured in the invention can have a chemical modification on a nucleotide in an internal (i.e., non-terminal) region having nencomplementarity with the target nucleic acid. For example, a modified nucleotide can be incorporated into the region of an miRNA that forms a bulge. The modification can include a ligand attached to the miRNA, e.g., by a linker. The modification can, for example, improve pharmacokinetics or stability of a therapeutic miRNA-type oligonucleotide agent, or improve hybridization properties (e.g., hybridization thermodynamics) of the miRNA-type oligonucleotide agent to a target nucleic acid. In some embodiments, it is preferred that the orientation of a modification or ligand incorporated into or tethered to the bulge region of an miRNA-type oligonucleotide agent is oriented to occupy the space in the bulge region. For example, the modification can include a modified base or sugar on the nucleic acid strand or a ligand that functions as an intercalator. These are preferably located in the bulge. The intercalator can be an aromatic, e.g., a polycyclic aromatic or heterocyclic aromatic compound. A polycyclic intercalator can have stacking capabilities, and can include systems with 2, 3, or 4 fused rings. The universal bases described below can be incorporated into the miRNA-type oligonucleotide agents. In some embodiments, it is preferred that the orientation of a modification or ligand incorporated into or tethered to the bulge region of an miRNA-type oligonucleotide agent is oriented to occupy the space in the bulge region. This orientation facilitates the improved hybridization properties or an otherwise desired characteristic of the miRNA-type oligonucleotide agent.

In one embodiment, an miRNA-type oligonucleotide agent or a pre-miRNA can include an aminoglycoside ligand, which can cause the miRNA-type oligonucleotide agent to have improved hybridization properties or improved sequence specificity. Exemplary aminoglycosides include glycosylated polylysine; galactosylated polylysine; neomycin B; tobramycin; kanamycin A; and acridine conjugates of aminoglycosides, such as Neo-N-acridine, Neo-S-acridine, Neo-C-acridine. Tobra-N-acridine, and KanaA-N-acridine. Use of an acridine analog can increase sequence specificity. For example, neomycin B has a high affinity for RNA as compared to DNA, but low sequence-specificity. In some embodiments the guanidine analog (the guanidinoglycoside) of an aminoglycoside ligand is tethered to an oligonucleotide agent. In a guanidinoglycoside, the amine group on the amino acid is exchanged for a guanidine group. Attachment of a guanidine analog can enhance cell permeability of an oligonucleotide agent.

In one embodiment, the ligand can include a cleaving group that contributes to target gene inhibition by cleavage of the target nucleic acid. Preferably, the cleaving group is tethered to the miRNA-type oligonucleotide agent in a manner such that it is positioned in the bulge region, where it can access and cleave the target RNA. The cleaving group can be, for example, a bleomycin (e.g., bleomycin-A₅, bleomycin-A₂, or bleomycin-B₂), pyrene, phenanthroline (e.g., O-phenanthroline), a polyamine, a tripeptide (e.g., lys-tyr-lys tripeptide), or metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to an miRNA or a pre-miRNA to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. The methods and compositions featured in the invention include miRNA-type oligonucleotide agents that inhibit target gene expression by a cleavage or non-cleavage dependent mechanism.

An miRNA-type oligonucleotide agent or pre-miRNA-type oligonucleotide agent can be designed and synthesized to include a region of noncomplementarity (e.g., a region that is 3, 4, 5, or 6 nucleotides long) flanked by regions of sufficient complementarity to form a duplex (e.g., regions that are 7, 8, 9, 10, or 11 nucleotides long) with a target RNA, e.g., an oligonucleotide agent, such as miR-122, miR-16, miR-192, or miR-194.

For increased nuclease resistance and/or binding affinity to the target, the single-stranded oligonucleotide agents featured in the invention can include 2′-O-methyl, 2′-fluorine, 2′-O-triethoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to the target. The inclusion of pyranose sugars in the oligonucleotide backbone can also decrease endonucleolytic cleavage. An oligonucleotide agent featured in the invention, e.g., a supermir, can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

The 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, an oligonucleotide agent, such as a single-stranded oligonucleotide agent, includes a modification that improves targeting, e.g. a targeting modification described herein. Examples of modifications that target single-stranded oligonucleotide agents to particular cell types include carbohydrate sugars such as galactose, N-acetylgalactosamine, mannose; vitamins such as folates; other ligands such as RGDs and RGD mimics; and small molecules including naproxen, ibuprofen or other known protein-binding molecules.

An oligonucleotide agent, such as a single-stranded oligonucleotide agent, featured in the invention can be constructed using chemical synthesis and/or enzymatic ligation reactions using procedures known in the art. For example, an oligonucleotide agent can be chemically synthesized using naturally occurring nucleotides or variously modified nucleotides designed to increase the biological stability of the molecules or to increase the physical stability of the duplex formed between the oligonucleotide agent and target nucleic acids, e.g., phosphorothioate derivatives and acridine substituted nucleotides can be used. Other appropriate nucleic acid modifications are described herein. Alternatively, the oligonucleotide agent can be produced biologically using an expression vector into which a nucleic acid has been subcloned in an antisense orientation (i.e., RNA transcribed from the inserted nucleic acid will be of an antisense orientation to a target nucleic acid of interest (e.g., an RNA target sequence of an endogenous miRNA or pre-miRNA)).

Chemical Definitions

The term “halo” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). Alkyl and haloalkyl groups may be optionally inserted with O, N, or S. The terms “aralkyl” refers to an alkyl moiety in which an alkyl hydrogen atom is replaced by an aryl group. Aralkyl includes groups in which more than one hydrogen atom has been replaced by an aryl group. Examples of “aralkyl” include benzyl, 9-fluorenyl, benzhydryl, and trityl groups.

The term “alkenyl” refers to a straight or branched hydrocarbon chain containing 2-8 carbon atoms and characterized in having one or more double bonds. Examples of a typical alkenyl include, but not limited to, allyl, propenyl, 2-butenyl, 3-hexenyl and 3-octenyl groups. The term “alkynyl” refers to a straight or branched hydrocarbon chain containing 2-8 carbon atoms and characterized in having one or more triple bonds. Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The terms “alkylamino” and “dialkylamino” refer to —NH(alkyl) and —NH(alkyl)₂ radicals respectively. The term “aralkylamino” refers to a —NH(aralkyl) radical. The term “alkoxy” refers to an —O-alkyl radical, and the terms “cycloalkoxy” and “aralkoxy” refer to an —O-cycloalkyl and O-aralkyl radicals respectively. The term “siloxy” refers to a R₃SiO-radical. The term “mercapto” refers to an SH radical. The term “thioalkoxy” refers to an —S-alkyl radical.

The term “alkylene” refers to a divalent alkyl (i.e., —R—), e.g., —CH₂—, —CH₂CH₂—, and —CH₂CH₂CH₂—. The term “alkylenedioxo” refers to a divalent species of the structure —O—R—O—, in which R represents an alkylene.

The term “aryl” refers to an aromatic monocyclic, bicyclic, or tricyclic hydrocarbon ring system, wherein any ring atom can be substituted. Examples of aryl moieties include, but are not limited to, phenyl, naphthyl, anthracenyl, and pyrenyl.

The term “cycloalkyl” as employed herein includes saturated cyclic, bicyclic, tricyclic, or polycyclic hydrocarbon groups having 3 to 12 carbons, wherein any ring atom can be substituted. The cycloalkyl groups herein described may also contain fused rings. Fused rings are rings that share a common carbon-carbon bond or a common carbon atom (e.g., spiro-fused rings). Examples of cycloalkyl moieties include, but are not limited to, cyclohexyl, adamantyl, and norbornyl, and decalin.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heterocyclyl groups herein described may also contain fused rings. Fused rings are rings that share a common carbon-carbon bond or a common carbon atom (e.g., spiro-fused rings). Examples of heterocyclyl include, but are not limited to tetrahydrofuranyl, tetrahydropyranyl, morpholine, pyrrolinyl and pyrrolidinyl.

The term “cycloalkenyl” as employed herein includes partially unsaturated, nonaromatic, cyclic, bicyclic, tricyclic, or polycyclic hydrocarbon groups having 5 to 12 carbons, preferably 5 to 8 carbons, wherein any ring atom can be substituted. The cycloalkenyl groups herein described may also contain fused rings. Fused rings are rings that share a common carbon-carbon bond or a common carbon atom (e.g., spiro-fused rings). Examples of cycloalkenyl moieties include, but are not limited to cyclohexenyl, cyclohexadienyl, norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heterocycloalkenyl groups herein described may also contain fused rings. Fused rings are rings that share a common carbon-carbon bond or a common carbon atom (e.g., spiro-fused rings). Examples of heterocycloalkenyl include but are not limited to tetrahydropyridyl and dihydropyran.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heteroaryl groups herein described may also contain fused rings that share a common carbon-carbon bond.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The terms “adeninyl, cytosinyl, guaninyl, thyminyl, and uracilyl” and the like refer to radicals of adenine, cytosine, guanine, thymine, and uracil.

A “protected” moiety refers to a reactive functional group, e.g., a hydroxyl group or an amino group, or a class of molecules, e.g., sugars, having one or more functional groups, in which the reactivity of the functional group is temporarily blocked by the presence of an attached protecting group. Protecting groups useful for the monomers and methods described herein can be found, e.g., in Greene, T. W., Protective Groups in Organic Synthesis (John Wiley and Sons: New York), 1981, which is hereby incorporated by reference.

### Supermir Structure

A supermir, such as a single-stranded oligonucleotide agent, featured in the invention includes a region sufficient complementarity to the target nucleic acid (e.g., target mRNA sequence of an endogenous miRNA or pre-miRNA), and is of sufficient length in terms of nucleotides, such that the supermir forms a duplex with the target nucleic acid. The supermir can modulate the function of the targeted molecule. For example, when the targeted molecule is an, RNA, such as mRNA targeted by an miRNA, e.g., miR-122, miR-16, miR-192, or miR-194, the supermir can supplement the gene silencing activity of the miRNA, which action will down-regulate expression of the mRNA targeted by the target miRNA. When the target is an mRNA, the supermir can replace or supplement the gene silencing activity of an endogenous miRNA.

For ease of exposition the term nucleotide or ribonucleotide is sometimes used herein in reference to one or more monomeric subunits of an oligonucleotide agent. It will be understood herein that the usage of the term “ribonucleotide” or “nucleotide” herein can, in the case of a modified RNA or nucleotide surrogate, also refer to a modified nucleotide, or surrogate replacement moiety at one or more positions.

A supermir featured in the invention is, or includes, a region that is at least partially, and in some embodiments fully, complementary to the target RNA. It is not necessary that there be perfect complementarity between the supermir and the target, but the correspondence must be sufficient to enable the oligonucleotide agent, or a cleavage product thereof, to modulate (e.g., inhibit) target gene expression.

A supermir will preferably have one or more of the following properties:
(1) it will be of the Formula 1, 2, 3, or 4 described below;
(2) it will have a 5′ modification that includes one or more phosphate groups or one or more analogs of a phosphate group;
(3) it will, despite modifications, even to a very large number of bases specifically base pair and form a duplex structure with a homologous target RNA of sufficient thermodynamic stability to allow modulation of the activity of the targeted RNA;
(4) it will, despite modifications, even to a very large number, or all of the nucleosides, still have “RNA-like” properties, i.e., it will possess the overall structural, chemical and physical properties of an RNA molecule, even though not exclusively, or even partly, of ribonucleotide-based content. For example, all of the nucleotide sugars can contain e.g., 2′OMe, 2′ fluoro in place of 2′ hydroxyl. This deoxyribonucleotide-containing agent can still be expected to exhibit RNA-like properties. While not wishing to be bound by theory, an electronegative fluorine prefers an axial orientation when attached to the C2′ position of ribose. This spatial preference of fluorine can, in turn, force the sugars to adopt a C_{3′}-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. (Generally, it is preferred that a modified moiety at the 2′ sugar position will be able to enter into hydrogen-bonding which is more characteristic of the 2′-OH moiety of a ribonucleotide than the 2′-H moiety of a deoxyribonucleotide. A preferred supermir will: exhibit a C_{3′}-endo pucker in all, or at least 50, 75, 80, 85, 90, or 95% of its sugars; exhibit a C_{3′}-endo pucker in a sufficient amount of its sugars that it can give rise to a the RNA-characteristic A-family-type helix; will have no more than 20, 10, 5, 4, 3, 2, or 1 sugar which is not a C_{3′}-endo pucker structure.

Preferred 2′-modifications with C3′-endo sugar pucker include:

2′-OH, 2′-O-Me, 2′-O-methoxyethyl, 2′-O-aminopropyl, 2′-F, 2′-O—CH2-CO—NHMe, 2′-O—CH2-CH2-O—CH2-CH2-N(Me)2, and LNA

Preferred 2′-modifications with a C2′-endo sugar pucker include:

2′-H, 2′-Me, 2′-S-Me, 2′-Ethynyl, 2′-ara-F.

Sugar modifications can also include L-sugars and 2′-5′-linked sugars.

As used herein, “specifically hybridizable” and “complementary” are terms that are used to indicate a sufficient degree of complementarity such that stable and specific binding occurs between a supermir of the invention and a target RNA molecule, e.g., an mRNA target of an endogenous miRNA or a pre-miRNA. Specific binding requires a sufficient lack of complementarity to non-target sequences under conditions in which specific binding is desired, i.e., under physiological conditions in the case of in viva assays or therapeutic treatment, or in the case of in vitro assays, under conditions in which the assays are performed. It has been shown that a single mismatch between targeted and non-targeted sequences are sufficient to provide discrimination for siRNA targeting of an mRNA (Brummelkamp et al., Cancer Cell, 2002, 2:243).

In one embodiment, an oligonucleotide agent featured in the invention, e.g., a supermir, is “sufficiently complementary” to a target RNA, such that the oligonucleotide agent inhibits production of protein encoded by the target mRNA. The target RNA can be, e.g., a pre-mRNA or mRNA endogenous to the subject. In another embodiment, the oligonucleotide agent is “exactly complementary” (excluding the SRMS containing subunit(s)) to a target RNA, e.g., the target RNA and the oligonucleotide agent can anneal to form a hybrid made exclusively of Watson-Crick base pairs in the region of exact complementarity. A “sufficiently complementary” target RNA can include a region (e.g., of at least 7 nucleotides) that is exactly complementary to a target RNA. Moreover, in some embodiments, the oligonucleotide agent specifically discriminates a single-nucleotide difference. In this case, the oligonucleotide agent only down-regulates gene expression if exact complementarity is found in the region of the single-nucleotide difference.

Oligonucleotide agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone. Examples of all of the above are discussed herein.

In some embodiments, the oligonucleotide agent is modified with one of the following modifications: modification of the nucleotide backbone such as modification of the phosphate linker or replacement of the phosphate linker; modification of the sugar moiety such as modification of the 2′ hydroxyl on the ribose; replacement of the sugar moiety such as ribose or deoxyribose with a different chemical structure such as a PNA structure; or modification of the nucleobase for example modification to a universal base or G-clamp.

As nucleic acids are polymers of subunits or monomers, many of the modifications described below occur at a position which is repeated within a nucleic acid, e.g., a modification of a base, or a phosphate moiety, or a non-linking O of a phosphate moiety. In some cases the modification will occur at all of the subject positions in the nucleic acid but in many, and in fact in most cases it will not. By way of example, a modification may only occur at a 3′ or 5′ terminal position, in a terminal region, e.g., at a position on a terminal nucleotide, or in the last 2, 3, 4, 5, or 10 nucleotides of a strand. The ligand can be attached at the 3′ end, the 5′ end, or at an internal position, or at a combination of these positions. For example, the ligand can be at the 3′ end and the 5′ end; at the 3′ end and at one or more internal positions; at the 5′ end and at one or more internal positions; or at the 3′ end, the 5′ end, and at one or more internal positions. For example, a phosphorothioate modification at a non-linking O position may only occur at one or both termini, or may only occur in a terminal region, e.g., at a position on a terminal nucleotide or in the last 2, 3, 4, 5, or 10 nucleotides of the oligonucleotide. The 5′ end can be phosphorylated.

Exemplary modifications of nucleotides are provided below:

Modifications and nucleotide surrogates are discussed below.

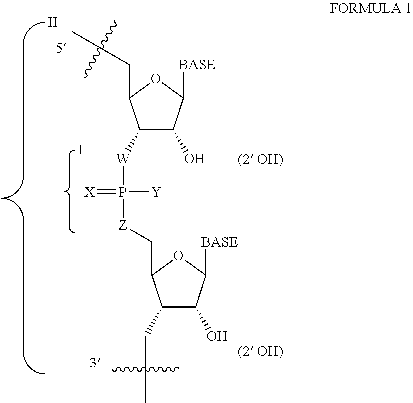

The scaffold presented above in Formula 1 represents a portion of a ribonucleic acid. The basic components are the ribose sugar, the base, the terminal phosphates, and phosphate internucleotide linkers. Where the bases are naturally occurring bases, e.g., adenine, uracil, guanine or cytosine, the sugars are the unmodified 2′ hydroxyl ribose sugar (as depicted) and W, X, Y, and Z are all O, Formula 1 represents a naturally occurring unmodified oligoribonucleotide.

Unmodified oligoribonucleotides may be less than optimal in some applications, e.g., unmodified oligoribonucleotides can be prone to degradation by e.g., cellular nucleases. Nucleases can hydrolyze nucleic acid phosphodiester bonds. However, chemical modifications to one or more of the above RNA components can confer improved properties, and, for example, can render oligoribonucleotides more stable to nucleases. Unmodified oligoribonucleotides may also be less than optimal in terms of offering tethering points for attaching ligands or other moieties to an oligonucleotide agent.

Modified nucleic acids and nucleotide surrogates can include one or more of:

(I) alteration, e.g., replacement, of one or both of the non-linking (X and Y) phosphate oxygens and/or of one or more of the linking (W and Z) phosphate oxygens (When the phosphate is in the terminal position, one of the positions W or Z will not link the phosphate to an additional element in a naturally occurring ribonucleic acid. However, for simplicity of terminology, except where otherwise noted, the W position at the 5′ end of a nucleic acid and the terminal Z position at the 3′ end of a nucleic acid, are within the term “linking phosphate oxygens” as used herein.);

(II) alteration, e.g., replacement, of a constituent of the ribose sugar, e.g., of the 2′ hydroxyl on the ribose sugar, or wholesale replacement of the ribose sugar with a structure other than ribose, e.g., as described herein;

(III) wholesale replacement of the phosphate moiety (bracket I) with “dephospho” linkers;

(IV) modification or replacement of a naturally occurring base;

(V) replacement or modification of the ribose-phosphate backbone (bracket II);

(VI) ligands.

The terms replacement, modification, alteration, and the like, as used in this context, do not imply any process limitation, e.g., modification does not mean that one must start with a reference or naturally occurring ribonucleic acid and modify it to produce a modified ribonucleic acid but rather modified simply indicates a difference from a naturally occurring molecule.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species. For example, any phosphoroamidate (replacement of a nonlinking oxygen with nitrogen) would be represented by X═O and Y═N in the above figure.

Specific modifications are discussed in more detail below.

(I) The Phosphate Group

The phosphate group is a negatively charged species. The charge is distributed equally over the two non-linking oxygen atoms (i.e., X and Y in Formula 1 above). However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. Unlike the situation where only one of X or Y is altered, the phosphorus center in the phosphorodithioates is achiral which precludes the formation of oligoribonucleotides diastereomers. Diastereomer formation can result in a preparation in which the individual diastereomers exhibit varying resistance to nucleases. Further, the hybridization affinity of RNA containing chiral phosphate groups can be lower relative to the corresponding unmodified RNA species. Thus, while not wishing to be bound by theory, modifications to both X and Y which eliminate the chiral center, e.g., phosphorodithioate formation, may be desirable in that they cannot produce diastereomer mixtures. Thus, X can be any one of S, Se, B, C, H, N, or OR (R is alkyl or aryl). Thus Y can be any one of S, Se, B, C, H, N, or OR (R is alkyl or aryl). Replacement of X and/or Y with sulfur is preferred. In some preferred embodiments, the phosphate is modified to a phosphorothioate, phosphorodithioate, boranophosphate, N3′-P5′ phosphoroamidate, thiophosphoroamidate, phosphoramidats, cationic phosphoramidate, phosphonoacetate, phosphonothioacetate, 3′-methylene phosphonate, or a methylphosphonate

The phosphate linker can also be modified by replacement of a linking oxygen (i.e., W or Z in Formula 1) with nitrogen (bridged phosphoroamidates), sulfur (bridged phosphorothioates) and carbon (bridged methylenephosphonates). The replacement can occur at a terminal oxygen (position W (3′) or position Z (5′)). Replacement of W with carbon or Z with nitrogen is preferred.

Exemplary modifications are also found in U.S. Ser. No. 11/170,798, which is incorporated herein by reference.

(II) The Sugar Group

A modified nucleotide agent can include modification of all or some of the sugar groups of the ribonucleic acid. For example, the 2′ hydroxyl group (OH) can be modified or replaced with a number of different “oxy” or “deoxy” substituents. While not being bound by theory, enhanced stability is expected since the hydroxyl can no longer be deprotonated to form a 2′ alkoxide ion. The 2′ alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge or ethylene bridge (e.g., 2′-4′-ethylene bridged nucleic acid (ENA)), to the 4′ carbon of the same ribose sugar; amino, O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂, CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Preferred substitutents are 2′-methoxyethyl, 2′-OCH3, 2′-O-allyl, 2′-C-allyl, and 2′-fluoro.

The sugar group can also contain one or more carbons that possess the opposite stereochemical configuration than that of the corresponding carbon in ribose. Thus, a modified RNA can include nucleotides containing e.g., arabinose, as the sugar.

Modified RNAs can also include “abasic” sugars, which lack a nucleobase at C-1′. These abasic sugars can also be further contain modifications at one or more of the constituent sugar atoms.

To maximize nuclease resistance, the 2′ modifications can be used in combination with one or more phosphate linker modifications (e.g., phosphorothioate). The so-called “chimeric” oligonucleotides are those that contain two or more different modifications.

The modification can also entail the wholesale replacement of a ribose structure with another entity (an SRMS) at one or more sites in the oligonucleotide agent.

In some preferred embodiments, the sugar modification includes one or more of 2′-OMe, 2′-F, 2′-O-MOE, 2′-O-thioMOE, 2′-O-AP, 2′-O-DMAOE, 2′-O-DMAEOE, 2′-O-NMA, 2′-O-DMAEA, 2′-O-GE, 2′-O-AE, 2′-O-DMAE, 2′-O-DMAP, 2′-O-ImBu, 2′-O-allyl, ANA, 2′-F-ANA, 3′-Modifications such as Terminal 3′-modifications, 2′-5′ linkages, 4′-Modifications, such as 4′-Thio sugar, 4′-F, 4′-C-aminoethyl, 5′-modifications, such as 5′-alkyl, O-alkyl, 5′-terminal modifications, such as 5′-hydroxymethyl, Bicyclic Sugars, LNA, ENA, α-L-LNA, and carbocyclic analogs of LNA.

In some preferred embodiments, the ribose is replaced with one or more of morpholino, a cationic Morpholino, a PNA, a PNA analog, HNA, or CeNA.

(III) Replacement of the Phosphate Group

The phosphate group can be replaced by non-phosphorus containing connectors (cf. Bracket 1 in Formula 1 above). While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

(IV) The Bases

Adenine, guanine, cytosine and uracil are the most common bases found in RNA. These bases can be modified or replaced to provide RNA's having improved properties. E.g., nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases, e.g., “unusual bases” and “universal bases” described herein, can be employed. Examples include without limitation 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia Of Polymer Science And Engineering, pages 858-859, Kroschwitz, 3. I., ed. John Wiley & Sons, 1990, and those disclosed by Englisch et al., Angewandte Chemie, to International Edition, 1991, 30, 613.

In some preferred embodiments, the nucleotide agent includes one or more of the following base modifications: C-5 modified pyrimidine, N-2 modified purine, N-6 modified purine, C-8 modified purine, 2,6-Diaminopurine, a universal base, G-clamp, phenoxazines, or thiophenoxazine.

Exemplary base modifications are described in U.S. Ser. No. 11/186,915; U.S. Ser. No. 11/197,753; and U.S. Ser. No. 11/119,533, each of which is incorporated herein by reference.

(V) Replacement of Ribophosphate Backbone

Oligonucleotide-mimicking scaffolds can also be constructed wherein the phosphate linker and ribose sugar are replaced by nuclease resistant nucleoside or nucleotide surrogates (see Bracket II of Formula 1 above). While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone.

Examples include the mophilino, cyclobutyl, pyrrolidine and peptide nucleic acid (PNA) nucleoside surrogates. A preferred surrogate is a PNA surrogate.

(VI) Ligands

An oligonucleotide agent can be modified to include a ligand. For example, the modification ban be at 3′ or 5′ ends of an oligonucleotide, or internally. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a spacer. The atom of the spacer can connect to or replace the linking atom of the phosphate group or the C-3′ or C-5′ O, N, S or C group of the sugar. Alternatively, the spacer can connect to or replace the atom of a nucleotide surrogate (e.g., PNAs). These spacers or linkers can include e.g., —(CH₂)ₙ—, —(CH₂)ₙN—, —(CH₂)ₙO—, —(CH₂)ₙS—O(CH₂CH₂O)ₙ, CH₂CH₂OH (e.g., n=3 or 6), abasic sugars, amide, carboxy, amine, oxyamine, oxyimine, thioether, disulfide, thiourea, sulfonamide, or molpholino, or biotin and fluorescein reagents. While not wishing to be bound by theory, it is believed that conjugation of certain moieties can improve transport, hybridization, and specificity properties. Again, while not wishing to be bound by theory, it may be desirable to introduce alterations (e.g., terminal alterations) that improve nuclease resistance. Other examples of terminal modifications include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Modifications (e.g., terminal modifications) can be added for a number of reasons, including as discussed elsewhere herein to modulate activity or to modulate resistance to degradation. Preferred modifications include the addition of a methylphosphonate at the 3′-most terminal linkage; a 3′ C5-aminoalkyl-dT; 3′ cationic group; or another 3′ conjugate to inhibit 3′-5′ exonucleolytic degradation.

Modifications (e.g., terminal modifications) useful for modulating activity include modification of the 5′ end with phosphate or phosphate analogs. E.g., in preferred embodiments oligonucleotide agents are 5′ phosphorylated or include a phosphoryl analog at the 5′ terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)₂(O)P-5′-CH2-), 5′-alkyletherphosphonates (R=alkylether=methoxymethyl (MeOCH2-), ethoxymethyl, etc., e.g. RP(OH)(O)—O-5′-).

Terminal modifications can also be useful for monitoring distribution, and in such cases the preferred groups to be added include fluorophores, e.g., fluorscein or an Alexa dye, e.g., Alexa 488. Terminal modifications can also be useful for enhancing uptake, useful modifications for this include cholesterol. Terminal modifications can also be useful for cross-linking anantagomir to another moiety; modifications useful for this include mitomycin C.

Exemplary lipophilic modifications include a cholesterol; a bile acid; and a fatty acid (e.g., lithocholic-oleyl, lauroyl, docosnyl, stearoyl, palmitoyl, myristoyl, oleoyl, linoleoyl). Other exemplary terminal modifications include the following, sugars, carbohydrates, folates and analogs thereof, PEGs, pluronics, PEI, endosomal releasing agents, cell surface targeting small molecules, cell surface targeting peptides (e.g. RGD and other phage display derived peptides), cell permeation peptides, nuclear targeting signal peptides (NLS), polymers (for example, polymers with targeting groups, polymers with endosomal releasing agents, polymers with biodegradable properties, polymers with nucleic acid packing groups (by charge intereactions, by hydrogen bonding interactions).

The above modifications can be made with the oligonucleotide agent with or without a linker (e.g., a cleavable linker), with or without a tether such as a long alkyl tether, with or without a spacer such as a PEG spacer, with or without a scaffold. The modifications can be made anywhere on the oligonucleotide agent, for example, at 5′-, 3′- or at internal positions.

Evaluation of Candidate Oligonucleotide Agents

One can evaluate a candidate single-stranded oligonucleotide agent, e.g., a modified candidate single-stranded oligonucleotide agent, for a selected property by exposing the agent or modified molecule and a control molecule to the appropriate conditions and evaluating for the presence of the selected property. For example, resistance to a degradent can be evaluated as follows. A candidate modified oligonucleotide agent, e.g., supermir, (and preferably a control single-stranded oligonucleotide agent, usually the unmodified form) can be exposed to degradative conditions, e.g., exposed to a milieu, which includes a degradative agent, e.g., a nuclease. For example, one can use a biological sample, e.g., one that is similar to a milieu, which might be encountered, in therapeutic use, e.g., blood or a cellular fraction, e.g., a cell-free homogenate or disrupted cells. The candidate and control can then be evaluated for resistance to degradation by any of a number of approaches. For example, the candidate and control could be labeled, preferably prior to exposure, with, e.g., a radioactive or enzymatic label, or a fluorescent label, such as Cy3 or Cy5. Control and modified oligonucleotide agents can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to inhibit gene expression. For example, a cell, e.g., a mammalian cell, such as a mouse or human cell, can be co-transfected with a plasmid expressing a fluorescent protein, e.g., GFP, and a candidate oligonucleotide agent homologous to the transcript encoding the fluorescent protein (see, e.g. WO 00/44914). For example, a modified oligonucleotide agent homologous to the GFP mRNA can be assayed for the ability to inhibit GFP expression by monitoring for a decrease in cell fluorescence, as compared to a control cell, in which the transfection did not include the candidate oligonucleotide agent, e.g., controls with no agent added and/or controls with a non-modified RNA added. Efficacy of the candidate agent on gene expression can be assessed by comparing cell fluorescence in the presence of the modified and unmodified oligonucleotide agent.

In an alternative functional assay, a candidate oligonucleotide agent homologous to an endogenous mouse gene, preferably a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by an oligonucleotide agent would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target RNA, or by Western blot to assay for a decrease in the level of target protein, as compared to a negative control. Controls can include cells in which with no agent is added and/or cells in which a non-modified RNA is added.

Preferred Oligonucleotide Agents

Preferred single-stranded oligonucleotide agents have the following structure (see Formula 2 below):

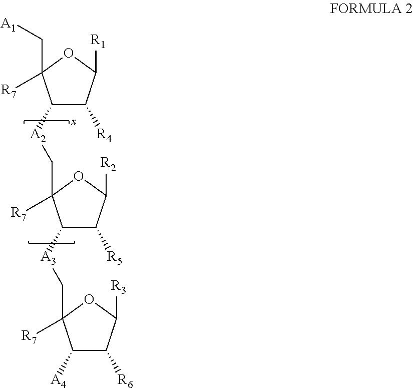

Referring to Formula 2 above, R¹, R², and R³ are each, independently, H, (i.e. abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil, substituted 1,2,4-triazoles, 2-pyridinone, 5-nitroindole, nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases.

R⁴, R⁵, and R⁶ are each, independently, OR⁸, O(CH₂CH₂O)ₘ, CH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂NHR⁹; NHC(O)R⁸; cyano; mercapto, SR⁸; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, or ureido; or R⁴, R⁵, or R⁶ together combine with R⁷ to form an [—O—CH₂-] covalently bound bridge between the sugar 2′ and 4′ carbons.

A¹ is:

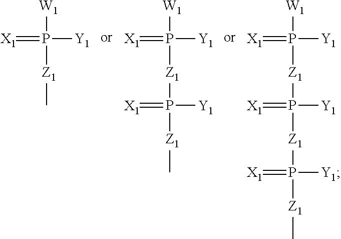

H; OH; OCH₃; W¹; an abasic nucleotide; or absent;

(a preferred A1, especially with regard to anti-sense strands, is chosen from 5′-monophosphate ((HO)₂(O)P—O-5′), 5′-diphosphate ((HO)₂(O))P—O—P(HO)(O)—O-5′), 5′-triphosphate ((HO)₂(O)P—O—(HO)(O)P—O—P(HO)(O)—O-5′), 5′-guanosine cap (7-methylated or non-methylated) (7m-G-O-5′-(HO)(O)P—O—(HO)(O)P—O—P(HO)(O)—O-5′), 5′-adenosine cap (Appp), and any modified or unmodified nucleotide cap structure (N—O-5′-(HO)(O)P—O—(HO)(O)P—O—P(HO)(O)—O-5′), 5′-monothiophosphate (phosphorothioate; (HO)₂(S)P—O-5′), 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, (OH)₂(O)P-5′-CH₂—), 5′-alkyletherphosphonates (R=alkylether=methoxymethyl (MeOCH₂—), ethoxymethyl, etc., e.g. RP(OH)(O)—O-5′-)).

A² is:

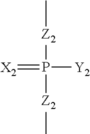

A³ is:

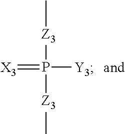

A⁴ is:

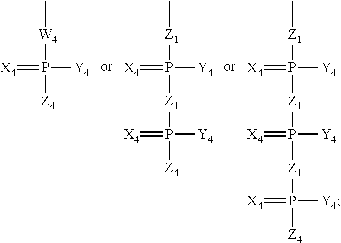

H; Z⁴; an inverted nucleotide; an abasic nucleotide; or absent.

W¹ is OH, (CH₂)ₙR¹⁰, (CH₂)ₙNHR¹⁰, (CH₂)ₙOR¹⁰, (CH₂)ₙSR¹⁰; O(CH₂)ₙR¹⁰; O(CH₂)ₙOR¹⁰, O(CH₂)ₙNR¹⁰, O(CH₂)ₙSR¹⁰; O(CH₂)ₙSS(CH₂)ₙOR¹⁰, O(CH₂)ₙC(O)OR¹⁰, NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰ O(CH₂CH₂O)ₘCH₂CH₂OR¹⁰; O(CH₂CH₂O)ₘCH₂CH₂NHR¹⁰, NH(CH₂CH₂NH)ₘCH₂CH₂NHR¹⁰; Q-R¹⁰, O-Q-R¹⁰ N-Q-R¹⁰, S-Q-R¹⁰ or —O—. W⁴ is O, CH₂, NH, or S.

X¹, X², X³, and X⁴ are each, independently, O or S.

Y¹, Y², Y³, and Y⁴ are each, independently, OH, O⁻, OR⁸, S, Se, BH₃⁻, H, NHR⁹, N(R⁹)₂ alkyl, cycloalkyl, aralkyl, aryl, or heteroaryl, each of which may be optionally substituted.

Z¹, Z², and Z³ are each independently O, CH₂, NH, or S. Z⁴ is OH, (CH₂)ₙR¹⁰, (CH₂)ₙNHR¹⁰, (CH₂)ₙOR¹⁰, (CH₂)ₙSR¹⁰; O(CH₂)ₙR¹⁰; O(CH₂)ₙOR¹⁰, O(CH₂)ₙNR¹⁰, O(CH₂)ₙSR¹⁰, O(CH₂)ₙSS(CH₂)ₙOR¹⁰, O(CH₂)ₙC(O)OR¹⁰; NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰ O(CH₂CH₂O)ₘCH₂CH₂OR¹⁰, O(CH₂CH₂O)ₘCH₂CH₂NHR¹⁰, NH(CH₂CH₂NH)ₘCH₂CH₂NHR¹⁰; Q-R¹⁰, O-Q-R¹⁰ N-Q-R¹⁰, S-Q-R¹⁰.

X is 5-100, chosen to comply with a length for an oligonucleotide agent described herein.

R⁷ is H; or is together combined with R⁴, R⁵, or R⁶ to form an [—O—CH₂-] covalently bound bridge between the sugar 2′ and 4′ carbons.

R⁸ is alkyl, cycloalkyl, aryl, aralkyl, heterocyclyl, heteroaryl, amino acid, or sugar; R⁹ is NH₂, alkylamino, dialkylamino, heterocyclyl, arylamino, diaryl amino, heteroaryl amino, diheteroaryl amino, or amino acid; and R¹⁰ is H; fluorophore (pyrene, TAMRA, fluorescein, Cy3 or Cy5 dyes); sulfur, silicon, boron or ester protecting group; intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (cholesterol, cholic acid, adamantine acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino; alkyl, cycloalkyl, aryl, aralkyl, heteroaryl; radiolabelled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles); or an oligonucleotide agent. M is 0-1,000,000, and n is 0-20. Q is a spacer selected from the group consisting of abasic sugar, amide, carboxy, oxyamine, oxyimine, thioether, disulfide, thiourea, sulfonamide, or morpholino, biotin or fluorescein reagents.

Preferred oligonucleotide agents in which the entire phosphate group has been replaced have the following structure (see Formula 3 below):

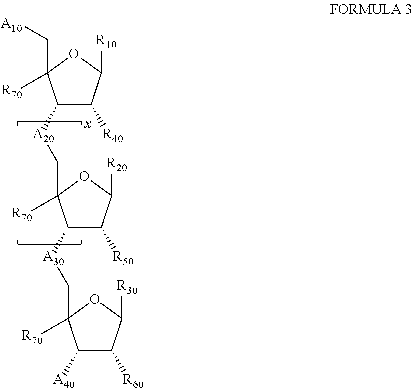

Referring to Formula 3. A¹⁰-A⁴⁰ is L-G-L; A¹⁰ and/or A⁴⁰ may be absent, in which L is a linker, wherein one or both L may be present or absent and is selected from the group consisting of CH₂(CH₂)_{g}; N(CH₂)_{g}; O(CH₂)_{g}; S(CH₂)_{g}. G is a functional group selected from the group consisting of siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

R¹⁰, R²⁰, and R³⁰ are each, independently, H, (i.e. abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases.

R⁴⁰, R⁵⁰, and R⁶⁰ are each, independently, OR⁸, O(CH₂CH₂O)ₘCH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂R⁹; NHC(O)R⁸; cyano; mercapto, SR⁷; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, acyloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, and ureido groups; or R⁴⁰, R⁵⁰, or R⁶⁰ together combine with R⁷⁰ to form an [—O—CH₂-] covalently bound bridge between the sugar 2′ and 4′ carbons.

X is 5-100 or chosen to comply with a length for an oligonucleotide agent described herein.

R⁷⁰ is H; or is together combined with R⁴⁰, R⁵⁰, or R⁶⁰ to form an [—O—CH₂—] covalently bound bridge between the sugar 2′ and 4′ carbons.

R⁸ is alkyl, cycloalkyl, aryl, aralkyl, heterocyclyl, heteroaryl, amino acid, or sugar; and R⁹ is NH₂, alkylamino, dialkylamino, heterocyclyl, arylamino, diaryl amino, heteroaryl amino, diheteroaryl amino, or amino acid. M is 0-1,000,000, n is 0-20, and g is 0-2.

Preferred nucleoside surrogates have the following structure (see Formula 4 below):

SLR¹⁰⁰-(M-SLR²⁰⁰)ₓ-M-SLR³⁰⁰ FORMULA 4

S is a nucleoside surrogate selected from the group consisting of mophilino, cyclobutyl, pyrrolidine and peptide nucleic acid. L is a linker and is selected from the group consisting of CH₂(CH₂)_{g}; N(CH₂)_{g}; O(CH₂)_{g}; S(CH₂)_{g}; —C(O)(CH₂)ₙ— or may be absent. M is an amide bond; sulfonamide; sulfinate; phosphate group; modified phosphate group as described herein; or may be absent.

R¹⁰⁰, R²⁰⁰, and R³⁰⁰ are each, independently, H (i.e., abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil substituted 1,2,4,-triazoles, 2-pyridinones, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyl adenine, N6-isopentyl adenine, 2-methylthio-N6-isopentenyl adenine, N-methylguanines, or O-alkylated bases.

X is 5-100, or chosen to comply with a length for an oligonucleotide agent described herein; and g is 0-2.

An oligonucleotide agent featured in the invention, e.g., a supermir, can include an internucleotide linkage (e.g., the chiral phosphorothioate linkage) useful for increasing nuclease resistance. In addition, or in the alternative, an oligonucleotide agent can include a ribose mimic for increased nuclease resistance. Exemplary internucleotide linkages and ribose mimics for increased nuclease resistance are described in co-owned PCT Application No. PCT/US2004/07070 filed on Mar. 8, 2004.

An oligonucleotide agent featured in the invention, e.g., a supermir, can include ligand-conjugated monomer subunits and monomers for oligonucleotide synthesis. Exemplary monomers are described in co-owned U.S. application Ser. No. 10/916,185, filed on Aug. 10, 2004.

An oligonucleotide agent featured in the invention, e.g., a supermir, can have a ZXY structure, such as is described in co-owned PCT Application No. PCT/US2004/07070 filed on Mar. 8, 2004.

An oligonucleotide agent featured in the invention, e.g., a supermir, can be complexed with an amphipathic moiety. Exemplary amphipathic moieties for use with oligonucleotide agents are described in co-owned PCT Application No. PCT/US2004/07070 filed on Mar. 8, 2004.

In another embodiment, the oligonucleotide agent featured in the invention, e.g., a supermir, can be complexed to a delivery agent that features a modular complex. The complex can include a carrier agent linked to one or more of (preferably two or more, more preferably all three of): (a) a condensing agent (e.g., an agent capable of attracting, e.g., binding, a nucleic acid, e.g., through ionic or electrostatic interactions); (b) a fusogenic agent (e.g., an agent capable of fusing and/or being transported through a cell membrane); and (c) a targeting group, e.g., a cell or tissue targeting agent, e.g., a lectin, glycoprotein, lipid or protein, e.g., an antibody, that binds to a specified cell type. oligonucleotide agents complexed to a delivery agent are described in co-owned PCT Application No. PCT/US2004/07070 filed on Mar. 8, 2004.

### Enhanced Nuclease Resistance

A supermir, such as a single-stranded oligonucleotide agent, featured in the invention can have enhanced resistance to nucleases.

For increased nuclease resistance and/or binding affinity to the target, an oligonucleotide agent, e.g., the oligonucleotide agent, can include, for example, 2′-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; amine, O-AMINE and aminoalkoxy, O(CH₂)ₙAMINE, (e.g., AMINE=NH₂; alkylamino, dialkylamino, heterocyclyl amino, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl amino, arylamino, diaryl amino, heteroaryl amino, or diheteroaryl amino), —NHC(O)R(R=alkyl, cycloalkyl, aryl, aralkyl, heteroaryl or sugar), cyano; mercapto; thioalkoxy; and alkyl, cycloalkyl, aryl, alkenyl and alkynyl, which may be optionally substituted with e.g., an amino functionality.

Preferred substitutents are 2′-methoxyethyl, 2′-OCH3, 2′-O-allyl, 2′-C-allyl, and 2′-fluoro.

One way to increase resistance is to identify cleavage sites and modify such sites to inhibit cleavage, as described in co-owned U.S. Application No. 60/559,917, filed on May 4, 2004. For example, the dinucleotides 5′-UA-3′,5′-UG-3′,5′-CA-3′,5′-UU-3′, or 5′-CC-3′ can serve as cleavage sites. Enhanced nuclease resistance can therefore be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. The oligonucleotide agent can include at least 2, at least 3, at least 4 or at least 5 of such dinucleotides. In certain embodiments, all the pyrimidines of an oligonucleotide agent carry a 2′-modification, and the oligonucleotide agent therefore has enhanced resistance to endonucleases.

To maximize nuclease resistance, the 2′ modifications can be used in combination with one or more phosphate linker modifications (e.g., phosphorothioate). The so-called “chimeric” oligonucleotides are those that contain two or more different modifications.

The inclusion of furanose sugars in the oligonucleotide backbone can also decrease endonucleolytic cleavage. An oligonucleotide agent can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′-end of oligonucleotide. Even small alkyl chains, aryl groups, or heterocyclic conjugates or modified sugars (D-ribose, deoxyribose, glucose etc.) can block 3′-5′-exonucleases.

Similarly, 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′-end of oligonucleotide. Even small alkyl chains, aryl groups, or heterocyclic conjugates or modified sugars (D-ribose, deoxyribose, glucose etc.) can block 3′-5′-exonucleases.

Thus, an oligonucleotide agent can include modifications so as to inhibit degradation, e.g., by nucleases, e.g., endonucleases or exonucleases, found in the body of a subject. These monomers are referred to herein as NRMs, or Nuclease Resistance promoting Monomers, the corresponding modifications as NRM modifications. In many cases these modifications will modulate other properties of the oligonucleotide agent as well, e.g., the ability to interact with a protein, e.g. a transport protein, e.g., serum albumin, or a member of the RISC, or the ability of the oligonucleotide agent to form a duplex with another sequence, e.g., a target molecule, such as an miRNA or pre-miRNA.

One or more different NRM modifications can be introduced into an oligonucleotide agent or into a sequence of an oligonucleotide agent. An NRM modification can be used more than once in a sequence or in an oligonucleotide agent.

NRM modifications include some which can be placed only at the terminus and others which can go at any position. Some NRM modifications that can inhibit hybridization are preferably used only in terminal regions, and more preferably not at the cleavage site or in the cleavage region of the oligonucleotide agent.

Modifications which interfere with or inhibit endonuclease cleavage should not be inserted in the region which is subject to RISC mediated cleavage, e.g., the cleavage site or the cleavage region (As described in Elbashir et al., Genes and Dev. 15: 188, 2001, hereby incorporated by reference). Cleavage of the target occurs about in the middle of a 20 or 21 nt oligonucleotide agent, or about 10 or 11 nucleotides upstream of the first nucleotide on the target mRNA which is complementary to the oligonucleotide agent. As used herein, cleavage site refers to the nucleotides on either side of the site of cleavage, on the target mRNA or on the oligonucleotide agent which hybridizes to it. Cleavage region means the nucleotides within 1, 2, or 3 nucleotides of the cleavage site, in either direction.

Such modifications can be introduced into the terminal regions, e.g., at the terminal position or with 2, 3, 4, or 5 positions of the terminus, of a sequence which targets or a sequence which does not target a sequence in the subject.

### Delivery of Single-Stranded Oligonucleotide Agents to Tissues and Cells

### Formulation

The single-stranded oligonucleotide agents described herein can be formulated for administration to a subject.

For ease of exposition, the formulations, compositions, and methods in this section are discussed largely with regard to unmodified oligonucleotide agents. It should be understood, however, that these formulations, compositions, and methods can be practiced with other oligonucleotide agents, e.g., modified oligonucleotide agents, and such practice is within the invention.

A formulated oligonucleotide agent featured in the invention, e.g., a supermir composition can assume a variety of states. In some examples, the composition is at least partially crystalline, uniformly crystalline, and/or anhydrous (e.g., less than 80, 50, 30, 20, or 10% water). In another example, the oligonucleotide agent is in an aqueous phase, e.g., in a solution that includes water, this form being the preferred form for administration via inhalation.

The aqueous phase or the crystalline compositions can be incorporated into a delivery vehicle, e.g., a liposome (particularly for the aqueous phase), or a particle (e.g., a microparticle as can be appropriate for a crystalline composition). Generally, the oligonucleotide agent composition is formulated in a manner that is compatible with the intended method of administration.

An oligonucleotide agent preparation can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the oligonucleotide agent preparation includes a second oligonucleotide agent, e.g., a second agent that can down-regulate expression of a second gene. Still other preparations can include at least three, five, ten, twenty, fifty, or a hundred or more different oligonucleotide species. In some embodiments, the agents are directed to the same target nucleic acid but different target sequences. In another embodiment, each oligonucleotide agent is directed to a different target. In one embodiment the oligonucleotide agent preparation includes a double stranded RNA that targets an RNA (e.g., an mRNA) for donwregulation by an RNAi silencing mechanism.

### Treatment Methods and Routes of Delivery

A composition that includes an agent featured in the invention, e.g., an agent that targets an miRNA or pre-miRNA (e.g., miR-122, miR-16, miR-192, miR-194, miR-141, mRR-143, miR-181, miR-181a, miR-181c, miR-192, miR-194, miR-200c, miR-206, miR-1, miR-205, miR-16, miR ebv-BHRF1-1, miR ebv-BHRF1-2, miR ebv-BHRF12-1, miR kshv-K3, miR kshv-K4-3p, miR kshv-mir-K2, miR kshv-mir-K5, miR kshv-mir-K6-3p, miR kshv-mir-K7, miR kshv-mir-K11, miR-31, miR-196, miR-215, miR-155, miR-142-5p, miR-142-3p, miR-143, Hsa-mir-146a, Hsa-mir-146b, mCMV-miR-01-1, mCMV-miR-01-2, mCMV-miR-23-1, mCMV-miR-23-2, mCMV-miR-44-1, miR-133, miR-133b, miR-124, miR-126, miR-126-3p, miR-126-5p, miR-21, miR-22, miR-122, miR-33) can be delivered to a subject by a variety of routes. Exemplary routes include inhalation, intrathecal, parenchymal, intravenous, nasal, oral, and ocular delivery.

An oligonucleotide featured in the invention, e.g., a supermir, can be incorporated into pharmaceutical compositions suitable for administration. For example, compositions can include one or more oligonucleotide agents and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions featured in the invention may be administered in a number of ways depending upon whether local or systemic treatment is desired and upon the area to be treated. Administration may be topical (including ophthalmic, intranasal, transdermal, intrapulmonary), oral or parenteral. Parenteral administration includes intravenous drip, subcutaneous, intraperitoneal or intramuscular injection, or intrathecal or intraventricular administration.

In general, delivery of an agent featured in the invention directs the agent to the site of infection in a subject. The preferred means of delivery is through local administration directly to the site of infection, or by systemic administration, e.g. parental administration.

Formulations for direct injection and parenteral administration are well known in the art. Such formulations may include sterile aqueous solutions which may also contain buffers, diluents and other suitable additives. For intravenous use, the total concentration of solutes should be controlled to render the preparation isotonic.

Administration of Oligonucleotide Agents A patient who has been diagnosed with a disorder characterized by unwanted miRNA expression (e.g., unwanted expression of miR-122, miR-16, miR-192, or miR-194) can be treated by administration of an oligonucleotide agent described herein to block the negative effects of the miRNA, thereby alleviating the symptoms associated with the unwanted miRNA expression. Similarly, a human who has or is at risk for deleveloping a disorder characterized by underexpression of a gene that is regulated by an miRNA can be treated by the administration of an oligonucleotide agent that targets the miRNA. For example, a human diagnosed with hemolytic anemia, and who carries a mutation in the aldolase A gene, expresses a compromised form of the enzyme. The patient can be administered an oligonucleotide agent that targets endogenous miR-122, which binds aldolase A RNA in vivo, presumably to downregulate translation of the aldolase A mRNA and consequently downregulate aldolase A protein levels. Administration of an oligonucleotide agent that targets the endogenous miR-122 in a patient having hemolytic anemia will decrease miR-122 activity, which will result in the upregulation of aldolase A expression and an increase in aldolase A protein levels. Although the enzyme activity of the mutant aldolase A is suboptimal, an increase in protein levels may be sufficient to relieve the disease symptoms. A human who has or who is at risk for developing arthrogryposis multiplex congenital, pituitary ectopia, rhabdomyolysis, or hyperkalemia, or who suffers from a myopathic symptom, is also a suitable candidate for treatment with an oligonucleotide agent that targets miR-122. A human who carries a mutation in the aldolase A gene can be a candidate for treatment with an oligonucleotide agent that targets miR-122. A human who carries a mutation in the aldolase A gene can have a symptom characterizing aldolase A deficiency including growth and developmental retardation, midfacial hypoplasia, and hepatomegaly.

In another example, a human who has or who is at risk for developing a disorder associated with overexpression of a gene regulated by an miRNA or by an miRNA deficiency, e.g., an miR-122, miR-192, or miR-194 deficiency, can be treated by the administration of an oligonucleotide agent, such as a single-stranded oligonucleotide agent, that is substantially identical to the deficient miRNA.

The single-stranded oligonucleotide agents featured in the invention can be administered systemically, e.g., orally or by intramuscular injection or by intravenous injection, in admixture with a pharmaceutically acceptable carrier adapted for the route of administration. An oligonucleotide agent can include a delivery vehicle, such as liposomes, for administration to a subject, carriers and diluents and their salts, and/or can be present in pharmaceutically acceptable formulations. Methods for the delivery of nucleic acid molecules are described in Akhtar et al., Trends in Cell Bio. 2:139, 1992; Delivery Strategies for Antisense Oligonucleotide Therapeutics, ed. Akhtar, 1995; Maurer et al., Mal. Membr. Biol., 16:129, 1999; Hofland and Huang, Handb. Exp. Pharmacol. 137:165, 1999; and Lee et al., ACS Symp. Ser. 752:184, 2000, all of which are incorporated herein by reference. Beigelman et al., U.S. Pat. No. 6,395,713 and Sullivan et al., PCT WO 94/02595 further describe the general methods for delivery of nucleic acid molecules. Nucleic acid molecules can be administered to cells by a variety of methods known to those of skill in the art, including, but not restricted to, encapsulation in liposomes, by ionophoresis, or by incorporation into other vehicles, such as hydrogels, cyclodextrins (see for example Gonzalez et al., Bioconjugate Chem. 10:1068, 1999), biodegradable nanocapsules, and bioadhesive microspheres, or by proteinaceous vectors (O'Hare and Normand, International PCT Publication No. WO 00/53722).

In the present methods, the oligonucleotide agent can be administered to the subject either as a naked oligonucleotide agent, in conjunction with a delivery reagent, or as a recombinant plasmid or viral vector which expresses the oligonucleotide agent. Preferably, the oligonucleotide agent is administered as a naked oligonucleotide agent.

An oligonucleotide agent featured in the invention can be administered to the subject by any means suitable for delivering the agent to the cells of the tissue at or near the area of unwanted target nucleic acid expression (e.g., target miRNA or pre-miRNA expression). For example, an oligonucleotide agent that targets miR-122 can be delivered directly to the liver, or can be conjugated to a molecule that targets the liver. Exemplary delivery methods include administration by gene gun, electroporation, or other suitable parenteral administration route.

Suitable enteral administration routes include oral delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application to the area at or near the site of neovascularization, for example by a catheter or other placement device (e.g., a retinal pellet or an implant comprising a porous, non-porous, or gelatinous material).

An oligonucleotide agent featured in the invention can be delivered using an intraocular implant. Such implants can be biodegradable and/or biocompatible implants, or may be non-biodegradable implants. The implants may be permeable or impermeable to the active agent, and may be inserted into a chamber of the eye, such as the anterior or posterior chambers, or may be implanted in the sclera, transchoroidal space, or an avascularized region exterior to the vitreous. In a preferred embodiment, the implant may be positioned over an avascular region, such as on the sclera, so as to allow for transscleral diffusion of the drug to the desired site of treatment, e.g., the intraocular space and macula of the eye. Furthermore, the site of transscleral diffusion is preferably in proximity to the macula.

An oligonucleotide agent featured in the invention can also be administered topically, for example, by patch or by direct application to the eye, or by iontophoresis. Ointments, sprays, or droppable liquids can be delivered by ocular delivery systems known in the art such as applicators or eyedroppers. The compositions can be administered directly to the surface of the eye or to the interior of the eyelid. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

An oligonucleotide agent featured in the invention may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

An oligonucleotide agent can be injected into the interior of the eye, such as with a needle or other delivery device.

An oligonucleotide agent featured in the invention can be administered in a single dose or in multiple doses. Where the administration of the oligonucleotide agent is by infusion, the infusion can be a single sustained dose or can be delivered by multiple infusions. Injection of the agent can be directly into the tissue at or near the site of aberrant or unwanted target gene expression (e.g., aberrant or unwanted miRNA or pre-miRNA expression). Multiple injections of the agent can be made into the tissue at or near the site.

Dosage levels on the order of about 1 μg/kg to 100 mg/kg of body weight per administration are useful in the treatment of a disease. One skilled in the art can also readily determine an appropriate dosage regimen for administering an oligonucleotide agent, e.g., a supermir, to a given subject. For example, the oligonucleotide agent can be administered to the subject once, e.g., as a single injection or deposition at or near the site on unwanted target nucleic acid expression. Alternatively, the oligonucleotide agent can be administered once or twice daily to a subject for a period of from about three to about twenty-eight days, more preferably from about seven to about ten days. In a preferred dosage regimen, the oligonucleotide agent is injected at or near a site of unwanted target nucleic acid expression once a day for seven days. Where a dosage regimen comprises multiple administrations, it is understood that the effective amount of an oligonucleotide agent administered to the subject can include the total amount of agent administered over the entire dosage regimen. One skilled in the art will appreciate that the exact individual dosages may be adjusted somewhat depending on a variety of factors, including the specific oligonucleotide agent being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known in the art. The precise therapeutically effective dosage levels and patterns are preferably determined by the attending physician in consideration of the above-identified factors.

In addition to treating pre-existing diseases or disorders, oligonucleotide agents featured in the invention (e.g., single-stranded oligonucleotide agents targeting miR-122, miR-16, miR-192, or miR-194) can be administered prophylactically in order to prevent or slow the onset of a particular disease or disorder. In prophylactic applications, an oligonucleotide agent is administered to a patient susceptible to or otherwise at risk of a particular disorder, such as disorder associated with aberrant or unwanted expression of an miRNA or pre-miRNA.

The oligonucleotide agents featured by the invention are preferably formulated as pharmaceutical compositions prior to administering to a subject, according to techniques known in the art. Pharmaceutical compositions featured in the present invention are characterized as being at least sterile and pyrogen-free. As used herein, “pharmaceutical formulations” include formulations for human and veterinary use. Methods for preparing pharmaceutical compositions are within the skill in the art, for example as described in Remington's Pharmaceutical Science, 18th ed., Mack Publishing Company, Easton, Pa. (1990), and The Science and Practice of Pharmacy. 2003, Gennaro et al., the entire disclosures of which are herein incorporated by reference.

The present pharmaceutical formulations include an oligonucleotide agent featured in the invention (e.g., 0.1 to 90% by weight), or a physiologically acceptable salt thereof, mixed with a physiologically acceptable carrier medium. Preferred physiologically acceptable carrier media are water, buffered water, normal saline, 0.4% saline, 0.3% glycine, hyaluronic acid and the like.

Pharmaceutical compositions featured in the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions can be packaged for use in liquid form, or can be lyophilized.

For solid compositions, conventional non-toxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25%-75%, of one or more single-stranded oligonucleotide agents featured in the invention.

By “pharmaceutically acceptable formulation” is meant a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as PluronicP85), which can enhance entry of drugs into the CNS (Jolliet-Riant and Tillement, Fundam. Clin. Pharmacol. 13:16, 1999); biodegradable polymers, such as poly (DL-lactide-coglycolide) microspheres for sustained release delivery. Other non-limiting examples of delivery strategies for the nucleic acid molecules featured in the instant invention include material described in Boado et al., J. Pharm. Sci. 87:1308, 1998; Tyler et al., FEBS Lett. 421:280, 1999; Pardridge et al., PNAS USA. 92:5592, 1995; Boado, Adv. Drug Delivery Rev. 15:73, 1995; Aldrian-Herrada et al., Nucleic Acids Res. 26:4910, 1998; and Tyler et al., PNAS USA 96:7053, 1999.

The invention also features the use of a composition that includes surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al., Chem. Rev. 95:2601, 1995; Ishiwata et al., Chem. Phare. Bull. 43:1005, 1995).

Such liposomes have been shown to accumulate selectively in tumors, presumably by extravasation and capture in the neovascularized target tissues (Lasic et al., Science 267:1275, 1995; Oku et al., Biochim. Biophys. Acta 1238:86, 1995). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 42:24864, 1995; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also features compositions prepared for storage or administration that include a pharmaceutically effective amount of the desired oligonucleotides in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

Alternatively, certain single-stranded oligonucleotide agents featured in the instant invention can be expressed within cells from eukaryotic promoters (e.g., Izant and Weintraub, Science 229:345, 1985; McGarry and Lindquist, Proc. Natl. Acad. Sei. USA 83:399, 1986; Scanlon et al., Proc. Natl. Acad. Sci. USA 88:10591, 1991; Kashani-Sabet et al., Antisense Res. Dev. 2:3, 1992; Dropulic et al., J. Virol. 66:1432, 1992; Weerasinghe et al., J. Virol. 65:5531, 1991; Ojwang et al., Proc. Natl. Acad. Sci. USA 89:10802, 1992; Chen et al., Nucleic Acids Res. 20:4581, 1992; Sarver et al., Science 247:1222, 1990; Thompson et al., Nucleic Acids Res. 23:2259, 1995; Good et al., Gene Therapy 4:45, 1997). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., Nucleic Acids Symp. Ser. 27:156, 1992; Taira et al., Nucleic Acids Res. 19:5125, 1991; Ventura et al., Nucleic Acids Res. 21:3249, 1993; Chowrira et al., J. Biol. Chem. 269:25856, 1994).

In another aspect of the invention, RNA molecules of the present invention can be expressed from transcription units (see for example Couture et al., Trends in Genetics 12:510, 1996) inserted into DNA or RNA vectors. The recombinant vectors can be DNA plasmids or viral vectors. Oligonucleotide agent-expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pats. Nos. 5,902,880 and 6,146,886). The recombinant vectors capable of expressing the oligonucleotide agents can be delivered as described above, and can persist in target cells. Alternatively, viral vectors can be used that provide for transient expression of nucleic acid molecules. Such vectors can be repeatedly administered as necessary. Once expressed, the oligonucleotide agent interacts with the target RNA (e.g., miRNA or pre-miRNA) and inhibits miRNA activity. In a preferred embodiment, the oligonucleotide agent forms a duplex with the target miRNA, which prevents the miRNA from binding to its target mRNA, which results in increased translation of the target mRNA. Delivery of oligonucleotide agent-expressing vectors can be systemic, such as by intravenous or intra-muscular administration, by administration to target cells ex-planted from a subject followed by reintroduction into the subject, or by any other means that would allow for introduction into the desired target cell (for a review see Couture et al., Trends in Genetics 12:510, 1996).

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the subject with no significant adverse toxicological effects on the subject.

The term “co-administration” refers to administering to a subject two or more single-stranded oligonucleotide agents. The agents can be contained in a single pharmaceutical composition and be administered at the same time, or the agents can be contained in separate formulation and administered serially to a subject. So long as the two agents can be detected in the subject at the same time, the two agents are said to be co-administered.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydmxypropyl-.beta.-cyclodextrin; and polysaccharides, such as raffinose, maltodextrins, dextrans, and the like; alditols, such as mannitol, xylitol, and the like. A preferred group of carbohydrates includes lactose, threhalose, raffinose maltodextrins, and mannitol. Suitable polypeptides include aspartame. Amino acids include alanine and glycine, with glycine being preferred.

Suitable pH adjusters or buffers include organic salts prepared from organic acids and bases, such as sodium citrate, sodium ascorbate, and the like; sodium citrate is preferred.

Dosage. An oligonucleotide agent featured in the invention, e.g., a supermir, can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of agent per kg of bodyweight. The unit dose, for example, can be administered by injection (e.g., intravenous or intramuscular, intrathecally, or directly into an organ), inhalation, or a topical application.

Delivery of an agent directly to an organ (e.g., directly to the liver) can be at a dosage on the order of about 0.00001 mg to about 3 mg per organ, or preferably about 0.0001-0.001 mg per organ, about 0.03-3.0 mg per organ, about 0.1-3.0 mg per organ or about 0.3-3.0 mg per organ.

The dosage can be an amount effective to treat or prevent a disease or disorder.

In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. Because oligonucleotide agent-mediated silencing can persist for several days after administering the oligonucleotide agent composition, in many instances, it is possible to administer the composition with a frequency of less than once per day, or, for some instances, only once for the entire therapeutic regimen.

In one embodiment, a subject is administered an initial dose, and one or more maintenance doses of an oligonucleotide agent. The maintenance dose or doses are generally lower than the initial dose, e.g., one-half less of the initial dose. A maintenance regimen can include treating the subject with a dose or doses ranging from 0.01 μg to 75 mg/kg of body weight per day, e.g., 70, 60, 50, 40, 30, 20, 10, 5, 2, 1, 0.5, 0.1, 0.05, 0.01, 0.005, 0.001, or 0.0005 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

Following successful treatment, it may be desirable to have the patient undergo maintenance therapy to prevent the recurrence of the disease state, wherein the compound of the invention is administered in maintenance doses, ranging from 0.01 μg to 100 g per kg of body weight (see U.S. Pat. No. 6,107,094).

The concentration of the oligonucleotide agent composition is an amount sufficient to be effective in treating or preventing a disorder or to regulate a physiological condition in humans. The concentration or amount of oligonucleotide agent administered will depend on the parameters determined for the agent and the method of administration, e.g. direct administration to the eye. For example, eye formulations tend to require much lower concentrations of some ingredients in order to avoid irritation or burning of the ocular tissues. It is sometimes desirable to dilute an oral formulation up to 10-100 times in order to provide a suitable ocular formulation.

Certain factors may influence the dosage required to effectively treat a subject, including but not limited to the severity of the disease or disorder, previous treatments, the general health and/or age of the subject, and other diseases present. It will also be appreciated that the effective dosage of the oligonucleotide agent used for treatment may increase or decrease over the course of a particular treatment. Changes in dosage may result and become apparent from the results of diagnostic assays. For example, the subject can be monitored after administering an composition containing the oligonucleotide agent. Based on information from the monitoring, an additional amount of the composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀s found to be effective in in vitro and in vivo animal models.

The invention is further illustrated by the following examples, which should not be construed as further limiting.

### EXAMPLES

### Example 1

### High Affinity Sugar-Base Modifications

Oligonucleotide Agents Containing High Affinity Nucleoside Modifications

High Affinity Sugar-Base Modifications

At least one of the listed nucleotide in Exemplification 2 is present in the oligonucleotide agent shown in Exemplification 1.

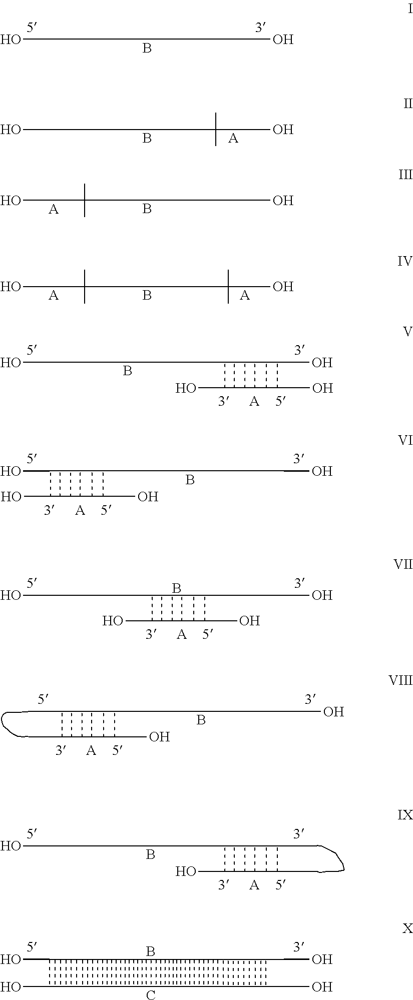

Exemplification 1. Oligonucleotide agent designs. I: Oligonucleotide agent; II: Oligonucleotide agent with 3′-ribosugar and phosphate (2-8 nucleotide in length); III: Oligonucleotide agent with 5′-ribosugar and phosphate (2-8 nucleotide in length); IV: Oligonucleotide agent with 3′ and 5′-ribosugar and phosphate (2-8 nucleotide in length); V: Oligonucleotide agent with 3′-end partial duplex with oligoribonucleotide (4-8 nucleotide in length); VI: Oligonucleotide agent with 5′-end partial duplex with oligoribonucleotide (4-8 nucleotide in length); VII: Oligonucleotide agent with internal partial duplex with oligoribonucleotide (4-8 nucleotide in length); VII: Oligonucleotide agent with 5′-end partial hairpin with oligoribonucleotide (4-8 nucleotide in length); IX: Oligonucleotide agent with 3′-end partial hairpin with oligoribonucleotide (4-8 nucleotide in length); X: Oligonucleotide agent with inactivated complementary antisense strand. Segment A indicates oligoribonucleotide with phosphate backbone; segment B indicates Oligonucleotide agent and segment C indicates inactivated antisense strand complementary to a Oligonucleotide agent.

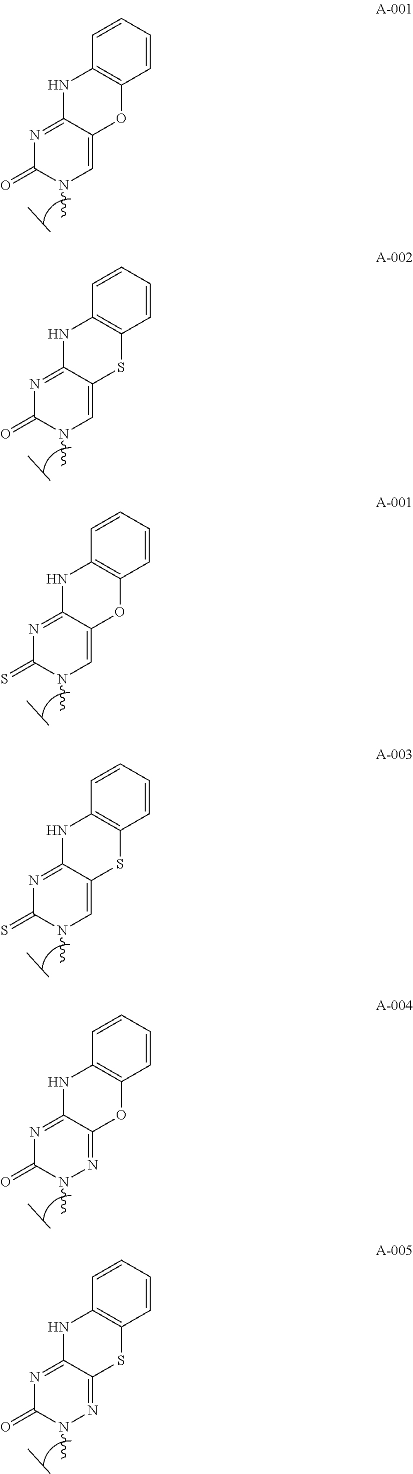

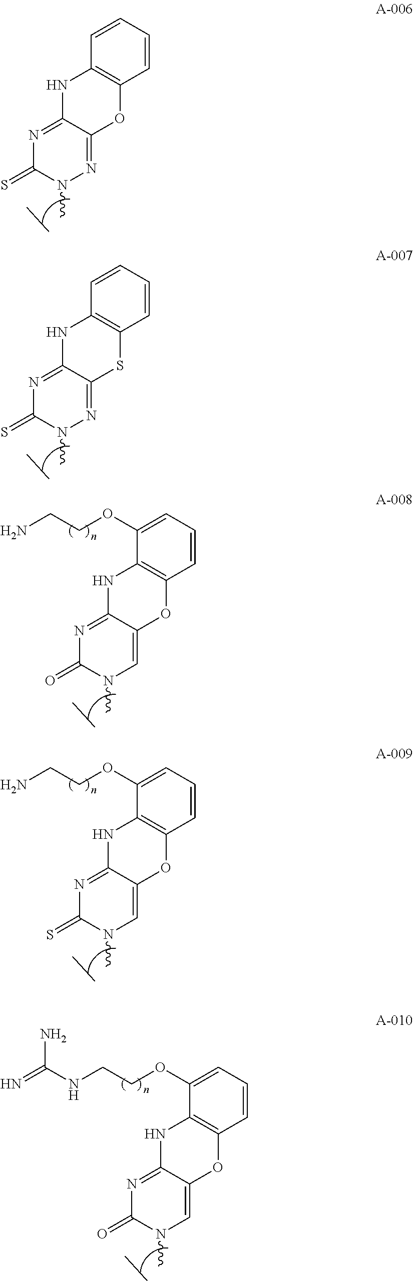

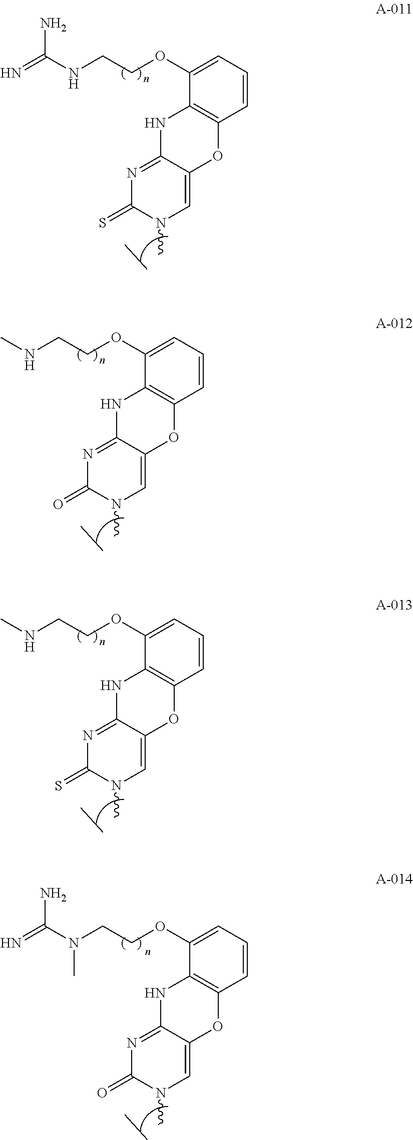

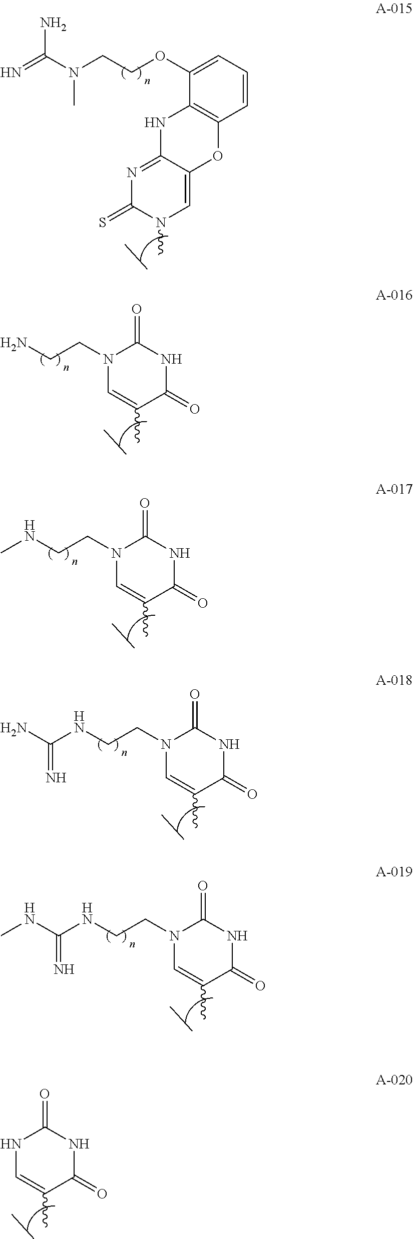

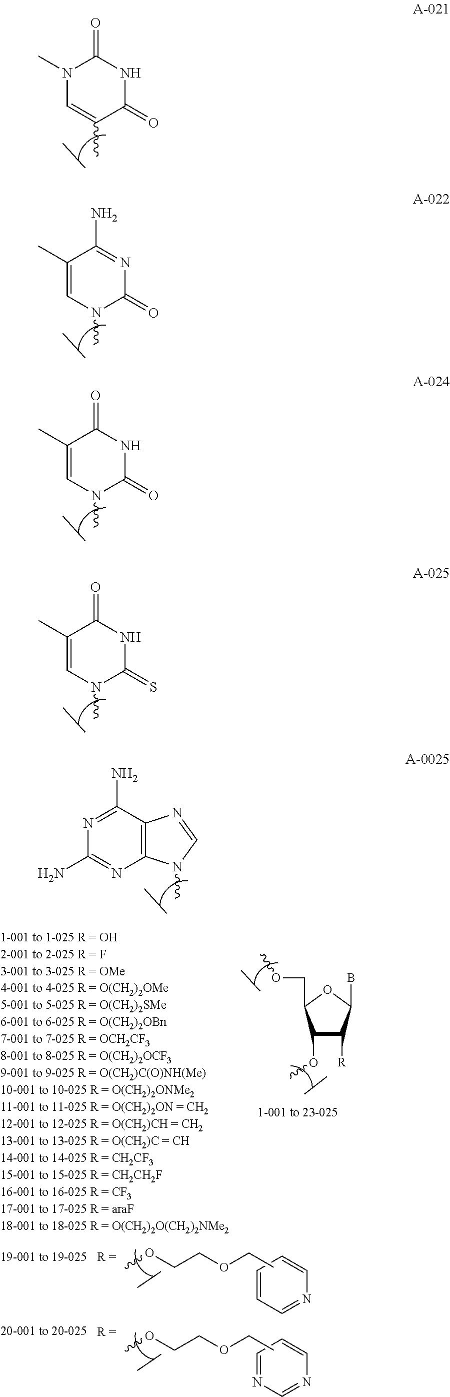

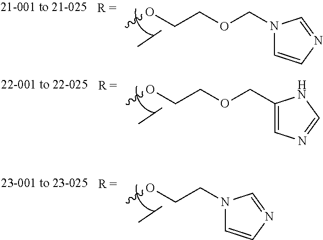

Exemplification 2. Compound 1-901 to 23-025 represents oligonucleotide agents with corresponding nucleoside modification. Lower case ‘n’=0-11 and uppercase A=1 to 24.

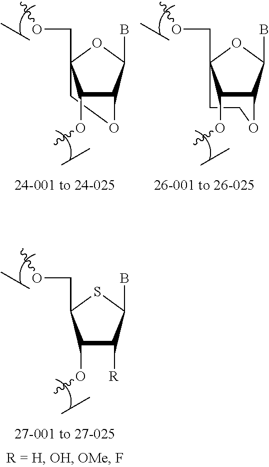

Exemplification 3. Sugar modifications. 24: LNA; 25: ENA and 26: 4′-Thio. B is from A-001 to A-025 of Exemplification 2.

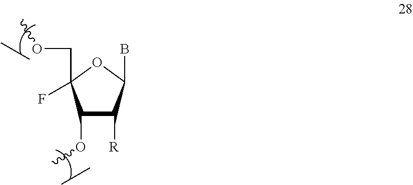

Exemplification 3. 4′-Fluoro sugar modification

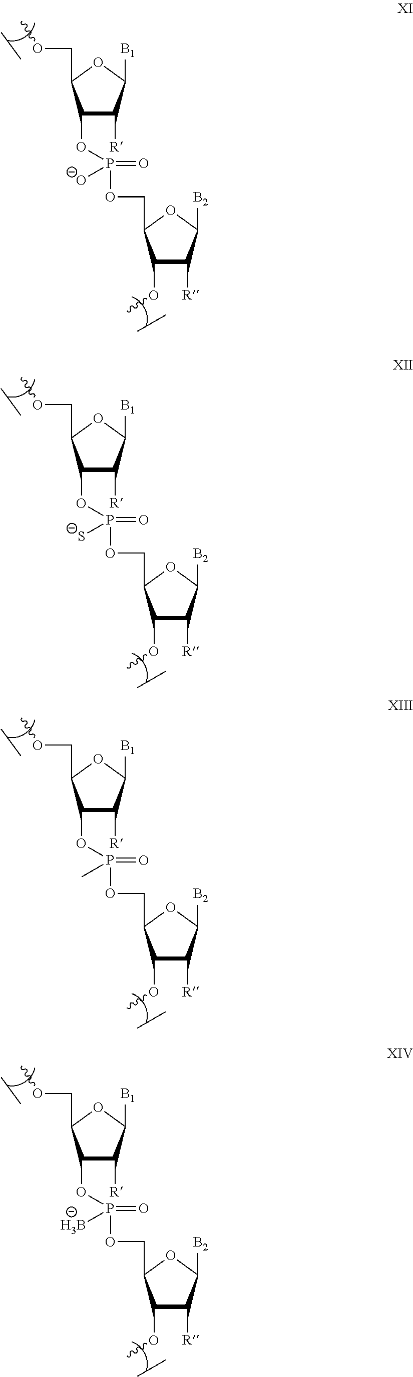

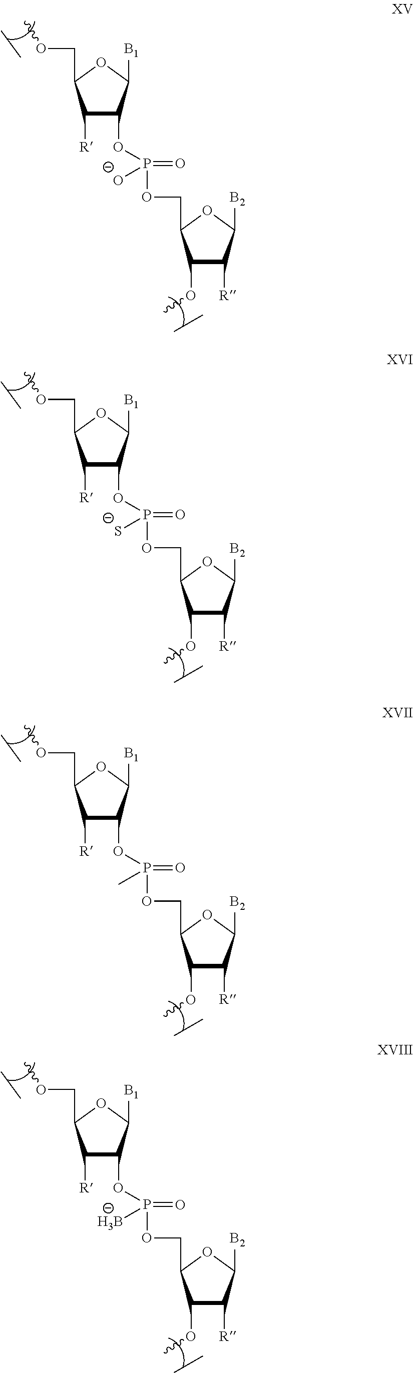

Exemplification 4. Backbone linkages. XI: 3′-5′ Phosphate; XII: 3′,5′ to phosphorothioate; XIII 3′,5′ methylphosphonate; XIV: 3′,5′ boranophosphate; Xv: 2′-5′Phosphate; XVI: 2′,5′ phosphorothioate; XVII 2′,5′ methylphosphonate; XVII: 2′,5′ boranophosphate

### Example 2

### High Affinity Oligonucleotides Containing 2-amino-2′-deoxy-2′-fluoro-adenosine

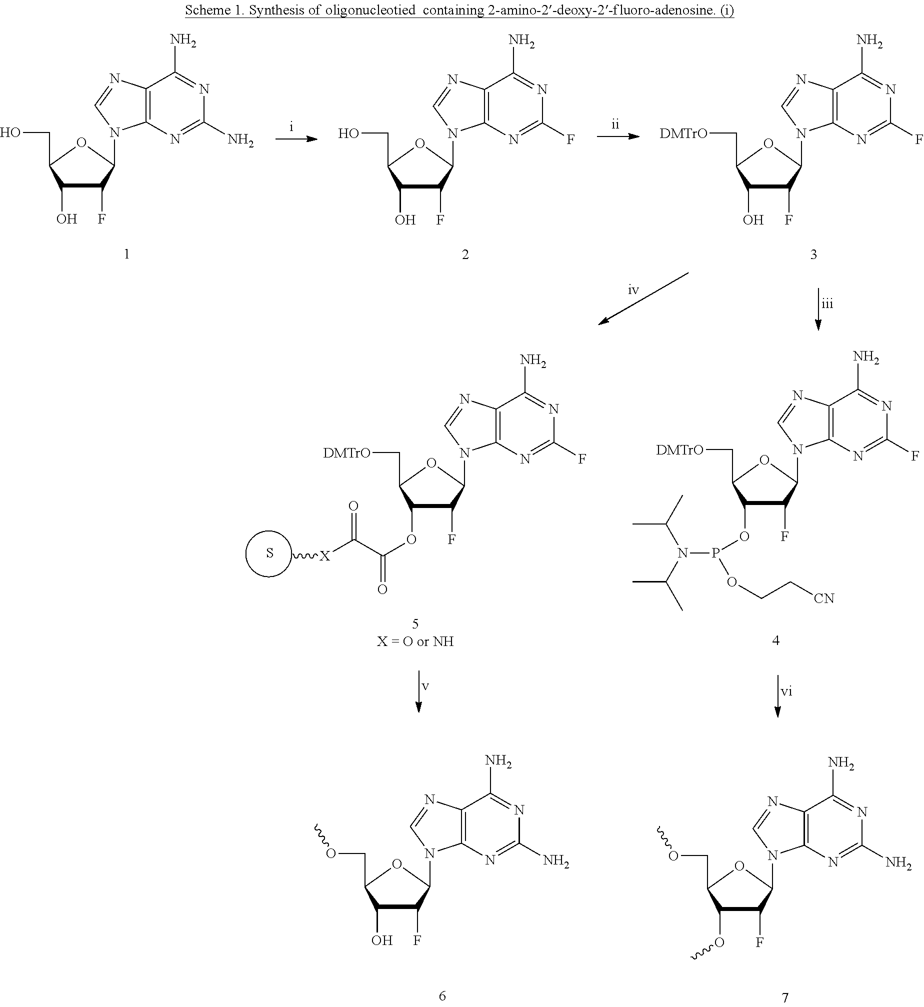

Step 1 Compound 2: A 2 L polyethylene bottle was equipped with a magnetic stirrer, thermometer, dry ice/acetone bath and a stream of argon gas. Anhydrous pyridine (500 mL) was added and the solution was cooled to −20° C. To this was added 70% hydrogen fluoride in pyridine (400 mL). 2′-fluoro-2,6-diaminopurine riboside (1, 90 g, 0.317 mol) was dissolved in the solution. Tert-butylnitrite (75 mL, 0.63 mol) was added in one portion and the reaction was stirred at 6-12° C. until the reaction was complete as judged by TLC (3 h). Sodium bicarbonate (2000 g) was suspended with manual stirring in water (2 L) in a 20 L bucket. The reaction solution was slowly poured (to allow for evolution of carbon dioxide) into the aqueous layer with vigorous stirring. The resulting solution was extracted with ethyl acetate (3×500 mL). The organic layers were combined and concentrated to a solid. The solid was mostly dissolved in methanol (300 mL) at reflux. The solution was cooled in a ice water bath and the resulting solid was collected, rinsed with methanol (2×50 mL) and dried under vacuum (1 mm Hg, 25° C., 24 h) to give 55 g of compound 2 as a dark gold solid. The mother liquor was concentrated and purified by column chromatography to give an additional 15.1 g of product for a total of 70.1 g (77%).

Step 2 Compound 3: 2,2′-Difluoro-2′-deoxyadenosine (2, 70 g, 0.244 mol) and dimethoxytrityl chloride (91.0 g, 0.268 mol) were dissolved in anhydrous pyridine (700 mL) and stirred at ambient temperature for 3 h. The reaction was quenched by the addition of methanol (50 mL) and then concentrated under reduced pressure to an oil. The residue was partitioned between ethyl acetate (1 L) and sat'd sodium bicarbonate (1 L mL). The aqueous layer was extracted with ethyl acetate (500 mL) once more and the combined extracts were concentrated under reduced pressure. The resulting solid was purified by crystallization from hexane-ethyl acetate (1:1) to give a light brown solid 3 (120.1 g, 83%).

Step 3 Compound 4: 5′-O-(4,4′-Dimethoxytrityl)-2,2′-difluoro-2′-deoxyadenosine (3, 90 g, 0.153 mol), 2-cyanoethyl tetraisopropylphosphorodiamidite (55.0 g, 0.183 mol), diisopropylamine tetrazolide (17.0 g, 0.1 mol) were dissolved in anhydrous dichloromethane (1 L) and allowed to stir at ambient temperature under an argon atmosphere for 16 h. The reaction was concentrated under reduced pressure to a thin oil and then directly applied to a silica gel column (200 g). The product was eluted with ethyl acetate-triethylamine (99:1). The appropriate fractions were combined, concentrated under reduced pressure, coevaporated with anhydrous acetonitrile and dried (1 mm Hg, 25° C., 24 h) to 109.1 g (90%) of light yellow foam of compound 4.

Step 4 Compound 5: 5′-O-(4,4′-Dimethoxytrityl)-2,2′-difluoro-2′-deoxyadenosine (4, 10 g, 16.9 mmol), dimethylaminopyridine (0.32 g, 2.6 mmol) and succinic anhydride (3.4 g, 34 mmol) were dissolved in anhydrous pyridine (50 mL) and stirred at ambient temperature under an argon atmosphere for 6 h. The reaction was quenched by the addition of water (20 mL) and then concentrated under reduced pressure to an oil. The oil was purified by column chromatography, eluted with methanol-dichloromethane-triethylamine (7:92:1). The appropriate fractions were collected and evaporated to give the product as a light yellow solid (10.4 g, 78%) of the corresponding succinate. The succinate is subsequently attached to solid support as reported in the literature to obtain the desired solid support 5.

Step 5 Compound 6: Oligonucleotide containing 2-Amino-2′-fluoro-adenosne at the 3′-end is synthesized starting from the solid support 5 according to the standard solid phase oligonucleotide synthesis and deprotection protocols. (Ref: WO00012563 and WO01002608). Oligonucleotide with phosphodiester and phosphorothioate backbone are prepared as reported by Ross et al. (WO00012563 and WO01002608)

Step 6 Compound 7: Oligonucleotide containing 2-amino-adenosine with 2′-deoxy-2′-fluoro sugar modification is synthesized and characterized as reported by Manoharan and Cook (Ref: WO01002608).

### Example 3

### High Affinity Oligonucleotides Containing Other 2-Amino-Adenosine Modifications

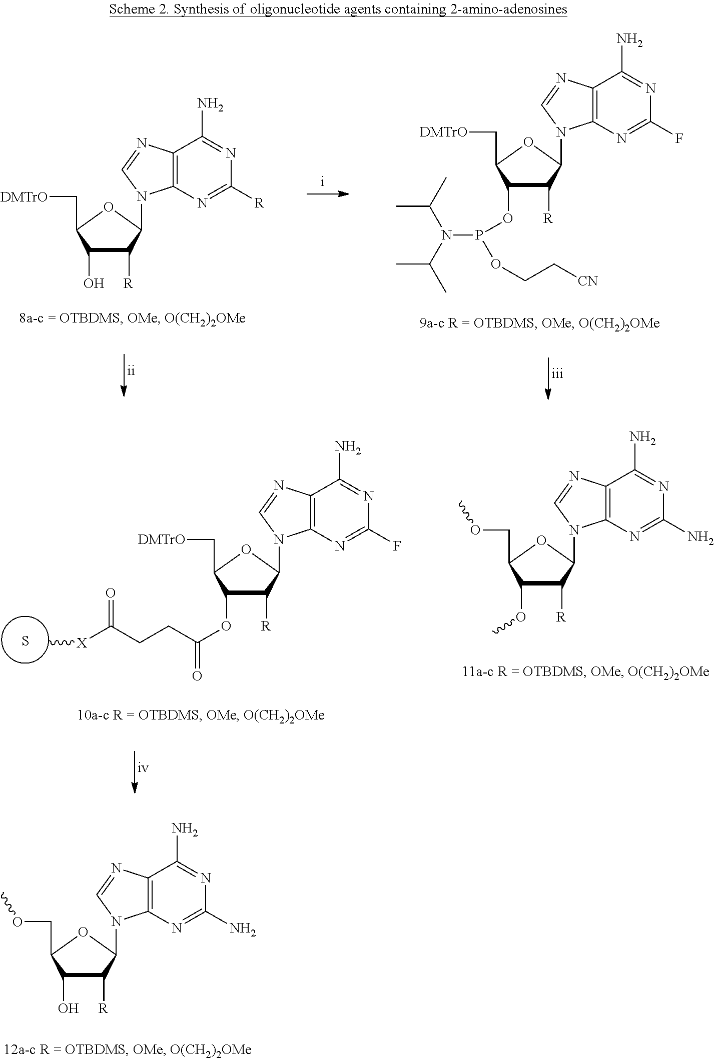

Compounds 8a-c are prepared as reported by Manoharan and Cook (Ref: WO01002608). Oligonucleotides with 2-amino-adenosine base modification (11a-c and 12a-c) are prepared as described in Example 1.

### Example 4

### High Affinity Oligonucleotides Containing High Affinity Phenoxazine and Thiophenoxazine

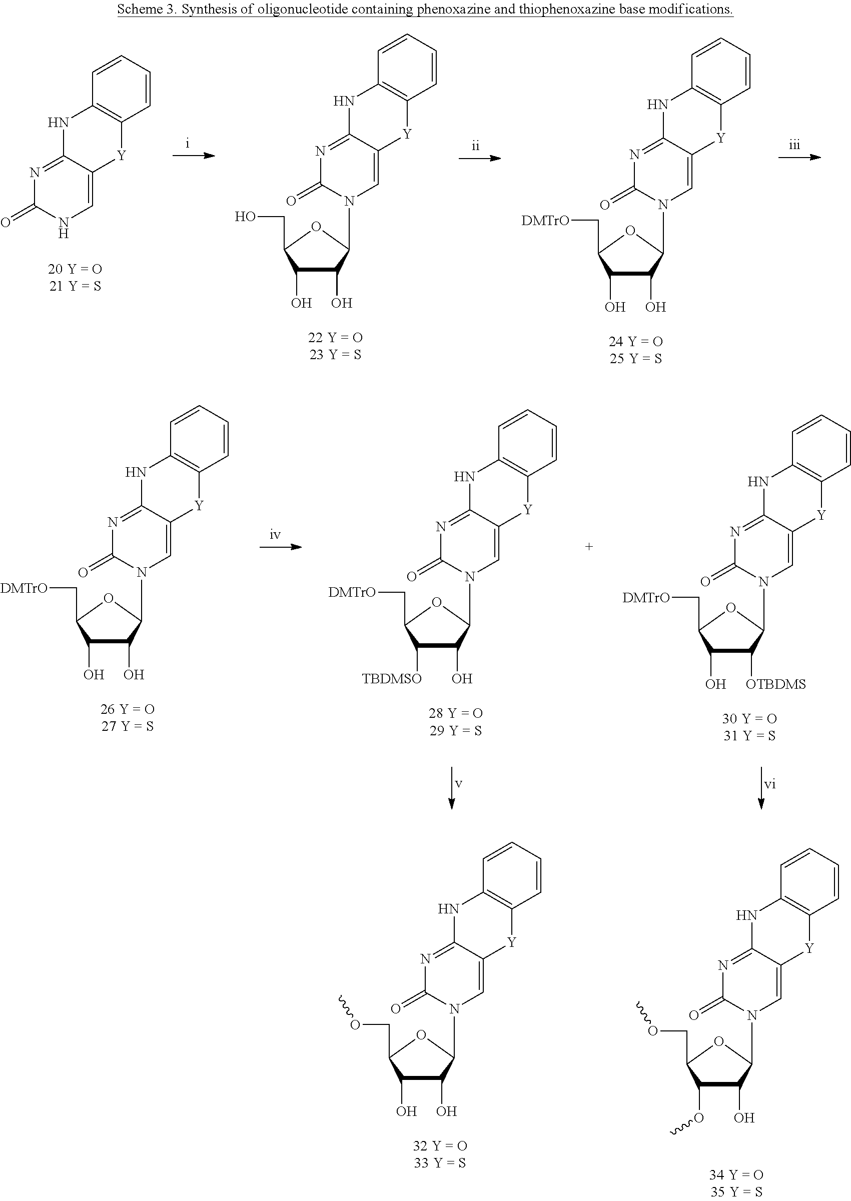

Step 1 Compounds 22 and 23: Compounds 20 and 21 are prepared according to the literature procedure (Sandin, Peter; Lincoln, Per; Brown, Tom; Wilhelmsson, L. Marcus. Nature Protocols, 2007, 2(3), 615-623). Nucleosides 22 and 23 are obtained respectively from 20 and 21 according to the procedures reported by Rajeev and Broom (Organic Lett., 2000, 2, 3595). Compounds 28 to 31 are obtained from respectively from 22 and 23 according to procedured reported to by Xia et al. (ACS Chem. Biol., 2006, 1, 176). Oligonucleotides 32 to 35 are synthesized from corresponding precursors 28 to 31 as described in Example 1 and as reported by Sandin et al. (Nature Protocols, 2007, 2(3), 615-623).

### Example 5

### High Affinity Oligonucleotides with G-Clamp Modification

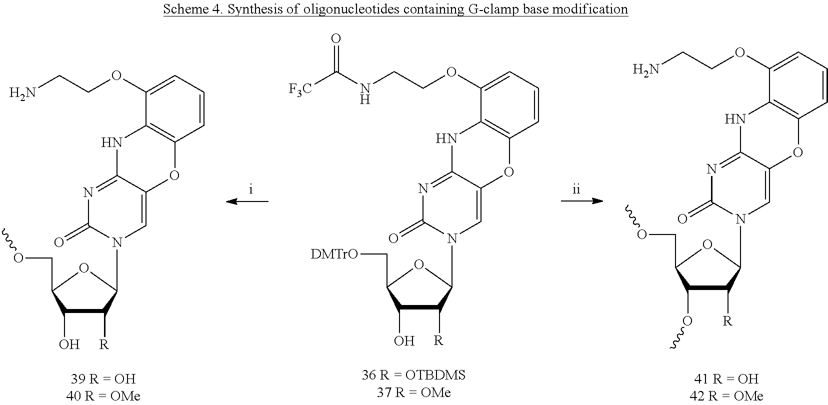

Compound 36 and 37 are prepared as reported by Holmes et al., Nucleic Acids Res., 2003, 31, 2759. Oligonucleotides 39 to 42 are obtained from corresponding precursors 36 and 37 as described in Examples 3 and 1.

### Example 6

### High Affinity Oligonucleotides Containing 2′-OMe and 2′-deoxy-2′-F Sugar Modified Phenoxazine Nucleosides

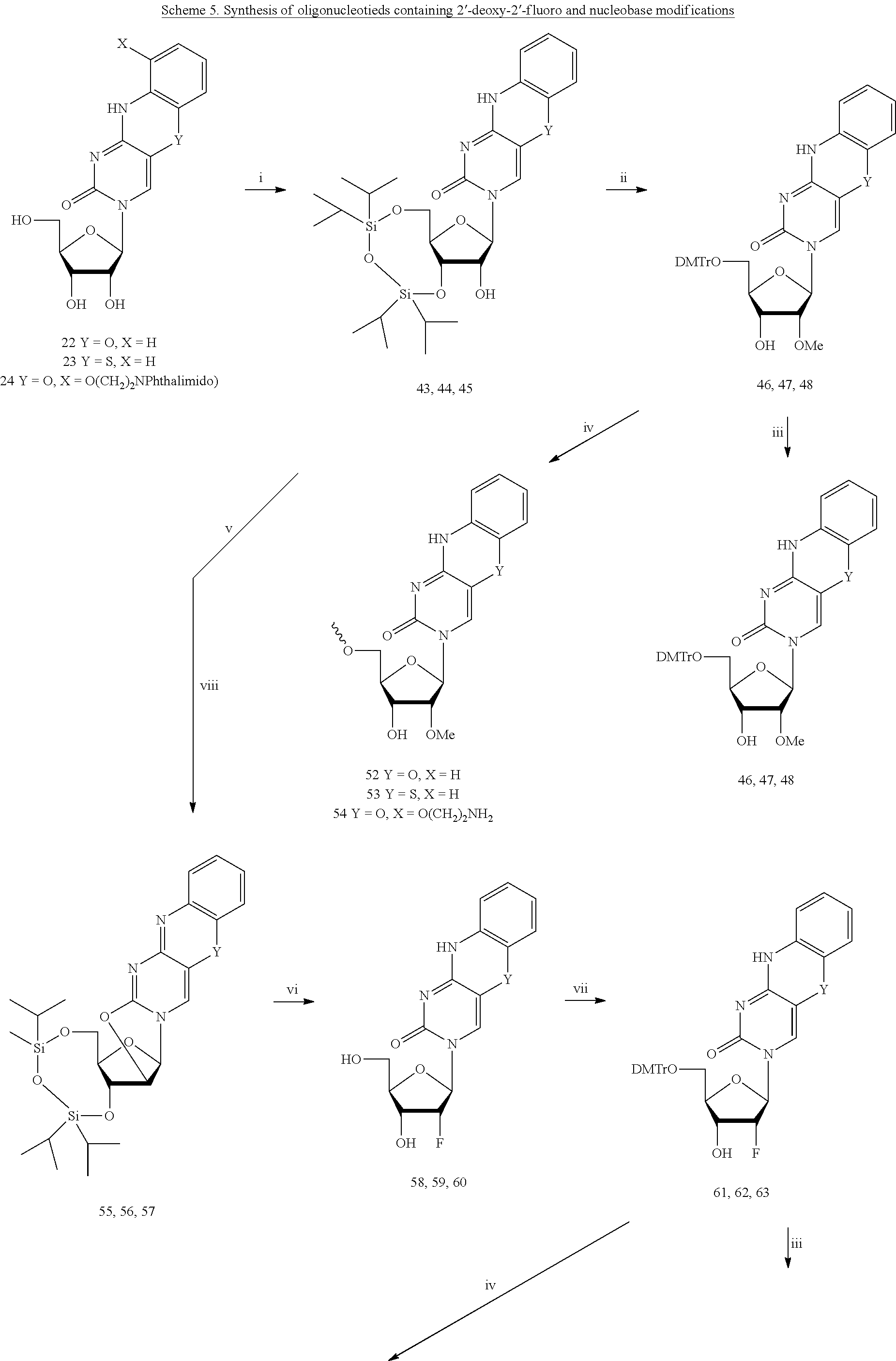

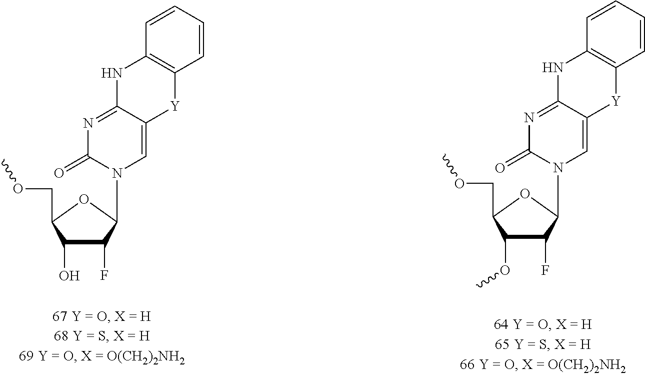

Compounds 46, 47 and 48 are prepared according to reported procedures (Holmes et al., Nucleic Acids Res., 2003, 31, 2759). Oligonucleotides 49 to 54 are obtained from corresponding precursors 47, 47 and 48 as described in Example 1

Compounds 58, 59 and 60 are prepared according to reported procedures (Shi et al. Bioorg. Med. Chem., 2005, 13, 1641). Oligonucleotides 64 to 69 are obtained from corresponding precursors 58, 59 and 60 as described in Example 1

### Example 6

### High Affinity Oligonucleotides Containing Pseudouridine Base Modifications

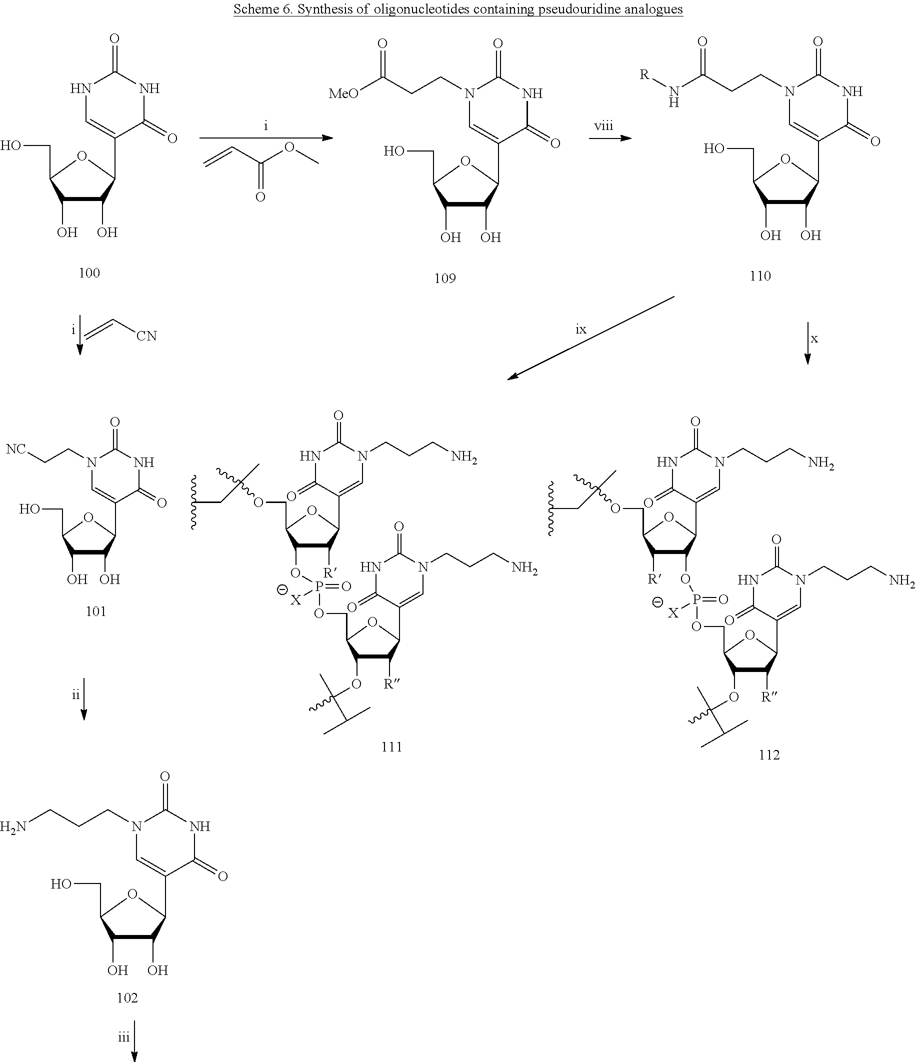

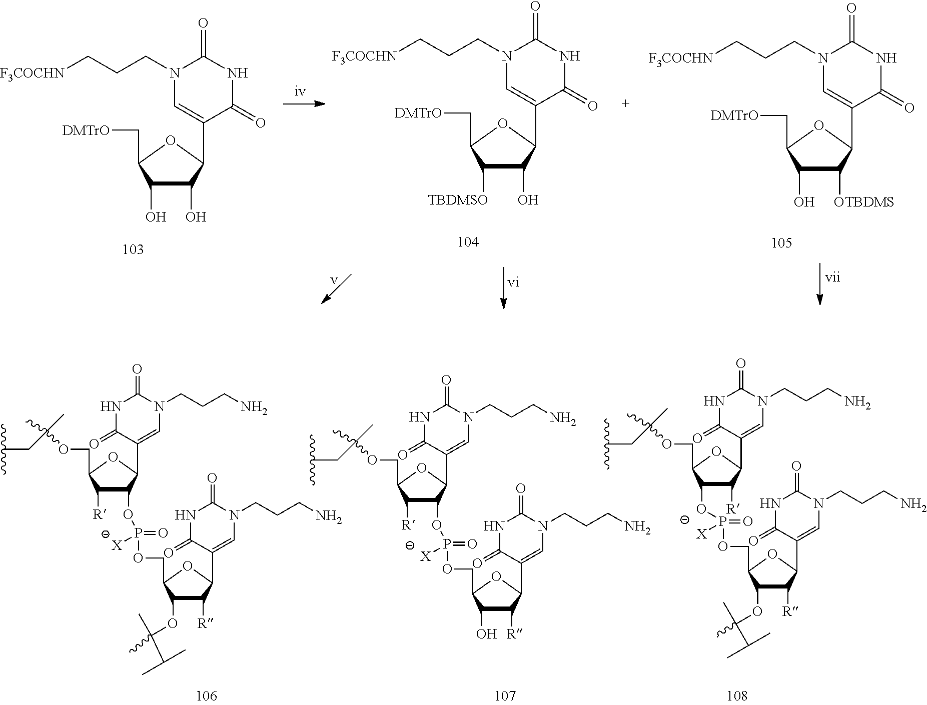

### Example 7

### Synthesis of Supermirs

Step 1. Oligonucleotide Synthesis

All oligonucleotides were synthesized on an AKTAoligopilot synthesizer or on an ABI 394 DNA/RNA synthesizer. Commercially available controlled pore glass solid supports (rU-CPG, 2′-O-methly modified rA-CPG and 2′-O-methyl modified rG-CPG from Prime Synthesis) or the in-house synthesized solid support hydroxyprolinol-cholesterol-CPG (described in patent xxxx) were used for the synthesis. RNA phosphoramidites and 2′-O-methyl modified RNA phosphoramidites with standard protecting groups (5′-O-dimethoxytrityl-N6-benzoyl-2′-t-butyldimethylsilyl-adenosine-3′-O—N,N′-diisopropyl-2-cyanoethylphosphorarnidite, 5′-O-dimethoxytrityl-N4-acetyl-2′-t-butyldimethylsilyl-cytidine-3′-O—N,N′-diisopropyl-2-cyanoethylphosphoramidite, 5′-O-dimethoxytrityl-N2-isobutryl-2′-t-butyldimethylsilyl-guanosine-3′-O—N,N′-diisopropyl-2-cyanoethylphosphoramidite, 5′-O-dimethoxytrityl-2′-t-butyldimethylsilyl-uridine-3′-O—N,N′-diisopropyl-2-cyanoethylphosphoramidite, 5′-O-dimethoxytrityl-N6-benzoyl-2′-O-methyl-adenosine-3′-O—N,N′-diisopropyl-2-cyanoethylphosphoramidite, 5′-O-dimethoxytrityl-N4-acetyl-2′-O-methyl-cytidine-3′-O—N,N′-diisopropyl-2-cyanoethylphosphoramidite, 5′-O-dimethoxytrityl-N2-isobutryl-2′-O-methyl-guanosine-3′-O—N,N′-diisopropyl-2-cyanoethylphosphoramidite, 5′-O-dimethoxytrityl-2′-O-methyl-uridine-3′-O—N,N′-diisopropyl-2-cyanoethylphosphoramidite and 5′-O-dimethoxytrityl-2′-deoxy-thymidine-3′-O—N,N′-diisopropyl-2-cyanoethylphosphoramidite) were obtained from Pierce Nucleic Acids Technologies and ChemGenes Research. The Quasar 570 phosphoramidite was obtained from Biosearch Technologies. The 5′-O-dimethoxytrityl-2′-t-butyldimethylsilyl-inosine-3′-O—N,N′-diisopropyl-2-cyanoethylphosphoramidite was obtained from ChemGenes Research. The 5′-O-dimethoxytrityl-2′-t-butyldimethylsilyl-(2,4)-difluorotolyl-3′-O—N,N′-diisopropyl-2-cyanoethylphosphoramidite (DFT-phosphoramidite) and the 5′-O-dimethoxytrityl-2′-t-butyldimethylsilyl-9-(2-aminoethoxy)-phenoxazine-3′-O—N,N′-diisopropyl-2-cyanoethylphosphoramidite (G-clamp phosphoramidite) were synthesized in house.

For the syntheses on AKTAoligopilot synthesizer, all phosphoramidites were used at a concentration of 0.2 M in CH₃CN except for guanosine and 2′-O-methyl-uridine, which were used at 0.2 M concentration in 10% THF/CH₃CN (v/v). Coupling/recycling time of 16 minutes was used for all phosphoramidite couplings. The activator was 5-ethyl-thio-tetrazole (0.75 M, American International Chemicals). For the PO-oxidation, 50 mM iodine in water/pyridine (10:90 v/v) was used and for the PS-oxidation 2% PADS (GL Synthesis) in 2,6-lutidine/CH₃CN (1:1 v/v) was used. For the syntheses on ABI 394 DNA/RNA synthesizer, all phosphoramidites, including DI-1 and G-clamp phosphoramidites were used at a concentration of 0.15 M in CH₃CN except for 2′-O-methyl-uridine, which was used at 0.15 M concentration in 10% THF/CH₃CN (v/v). Coupling time of 10 minutes was used for all phosphoramidite couplings. The activator was 5-ethyl-thio-tetrazole (0.25 M, Glen Research). For the PO-oxidation, 20 mM iodine in water/pyridine (Glen Research) was used and for the PS-oxidation 0.1M DDTT (AM Chemicals) in pyridine was used. Coupling of the Quasar 570 phosphoramidite was carried out on the ABI DNA/RNA synthesizer. The Quasar 570 phosphoramidite was used at a concentration of 0.1M in CH₃CN with a coupling time of 10 mins. The activator was 5-ethyl-thio-tetrazole (0.25 M, Glen Research) and 0.1M DDTT (AM Chemicals) in pyridine was used for PS oxidation.

Step 2. Deprotection of Oligonucleotides

A. Sequences Synthesized on the AKTAoligopilot Synthesizer

After completion of synthesis, the support was transferred to a 100 mL glass bottle (VWR). The oligonucleotide was cleaved from the support with simultaneous deprotection of base and phosphate groups with 40 mL of a 40% aq, methyl amine (Aldrich) 90 mins at 45° C. The bottle was cooled briefly on ice and then the methylamine was filtered into a new 500 mL bottle. The CPG was washed three times with 40 mL portions of DMSO. The mixture was then cooled on dry ice.

In order to remove the tert-butyldimethylsilyl (TBDMS) groups at the 2′ position, 60 mL triethylamine trihydrofluoride (Et3N—HF) was added to the above mixture. The mixture was heated at 40° C. for 60 minutes. The reaction was then quenched with 220 mL of 50 mM sodium acetate (pH 5.5) and stored in the freezer until purification.

B. Sequences Synthesized on the ABI DAN/RNA Synthesizer

After completion of synthesis, the support was transferred to a 15 mL tube (VWR). The oligonucleotide was cleaved from the support with simultaneous deprotection of base and phosphate groups with 7 mL of a 40% aq. methyl amine (Aldrich) 15 mins at 65° C. The bottle was cooled briefly on ice and then the methylamine was filtered into a 100 mL bottle (VWR). The CPG was washed three times with 7 mL portions of DMSO. The mixture was then cooled on dry ice.

In order to remove the ten-butyldimethylsilyl (TBDMS) groups at the 2′ position, 10.5 mL triethylamine trihydrofluoride (Et3N—HF) was added to the above mixture. The mixture was heated at 60° C. for 15 minutes. The reaction was then quenched with 38.5 mL of 50 mM sodium acetate (pH 5.5) and stored in the freezer until purification.

Step 3. Quantitation of Crude Oligonucleotides

For all samples, a 10 μL aliquot was diluted with 990 μL of deionised nuclease free water (1.0 mL) and the absorbance reading at 260 nm was obtained.

Step 4. Purification of Oligonucleotides

### (a) Unconjugated Oligonucleotides

The unconjugated crude oligonucleotides were first analyzed by HPLC (Dionex PA 100). The buffers were 20 nM phosphate, pH 11 (buffer A); and 20 mM phosphate, 1.8 M NaBr, pH 11 (buffer B). The flow rate 1.0 mL/min and monitored wavelength was 260-280 nm. Injections of 5-15 μL were done for each sample.

The unconjugated samples were purified by HPLC on a TSK-Gel SuperQ-5PW (20) column packed in house (17.3×5 cm) or on a commercially available TSK-Gel SuperQ-5PW column (15×0.215 cm) available from TOSOH Bioscience. The buffers were 20 mM phosphate in 10% CH₃CN, pH 8.5 (buffer A) and 20 mM phosphate, 1.0 M NaBr in 10% CH₃CN, pH 8.5 (buffer B). The flow rate was 50.0 ml/min for the in house packed column and 10.0 ml/min for the commercially obtained column. Wavelengths of 260 and 294 nm were monitored. The fractions containing the full-length oligonucleotides were pooled together, evaporated, and reconstituted to ˜100 mL with deionised water.

### (b) Cholesterol-Conjugated Oligonucleotides

The cholesterol-conjugated crude oligonucleotides were first analyzed by LC/MS to determine purity. The cholesterol conjugated sequences were HPLC purified on RPC-Source15 reverse-phase columns packed in house (17.3×5 cm or 15×2 cm). The buffers were 20 mM NaOAc in 10% CH₃CN (buffer A) and 20 mM NaOAc in 70% CH₃CN (buffer B). The flow rate was 50.0 mL/min for the 17.3×5 cm column and 12.0 ml/min for the 15×2 cm column. Wavelengths of 260 and 284 nm were monitored. The fractions containing the full-length oligonucleotides were pooled, evaporated, and reconstituted to 100 mL with deionised water.

Step 5. Desalting of Purified Oligonucleotides

The purified oligonucleotides were desalted on either an AKTA Explorer or an AKTA Prime system (Amersham Biosciences) using a Sephadex G-25 column packed in house. First, the column was washed with water at a flow rate of 40 mL/min for 20-30 min. The sample was then applied in 40-60 mL fractions. The eluted salt-free fractions were combined, dried, and reconstituted in ˜50 mL of RNase free water.

Step 6. Purity Analysis by Capillary Gel Electrophoresis (CGE), Ion-Exchange HPLC (IEX), and Electrospray LC/Ms

Approximately 0.3 OD of each of the desalted oligonucleotides were diluted in water to 300 μL and were analyzed by CGE, ion exchange HPLC, and LC/MS.

Step 7. Duplex Formation

For the fully double stranded duplexes, equal amounts, by weight, of two RNA strands were mixed together. The mixtures were frozen at −80° C. and dried under vacuum on a speed vac. Dried samples were then dissolved in 1×PBS to a final concentration of 40 mg/ml. The dissolved samples were heated to 95° C. for 5 min and slowly cooled to room temperature.

Step 8. Tm Determination

For the partial double stranded duplexes and hairpins melting temperatures were determined. For the duplexes, equimolar amounts of the two single stranded RNAs were mixed together. The mixtures were frozen at −80° C. and dried under vacuum on a speed vac. Dried samples were then dissolved in 1×PBS to a final concentration of 2.5 μM. The dissolved samples were heated to 95° C. for 5 min and slowly cooled to room temperature. Denaturation curves were acquired between 10-90° C. at 260 nm with temperature ramp of 0.5° C./min using a Beckman spectrophotometer fitted with a 6-sample thermostated cell block. The Tm was then determined using the 1st derivative method of the manufacturer's supplied program.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method of increasing the effect of a microRNA in a cell in a subject comprising the step of administering an agonist modified oligonucleotide agent to the subject, wherein the modified agonist oligonucleotide agent is substantially single-stranded, comprises a sequence which is substantially identical to antagomir 3547 miRNA.


SEQUENCE LISTING (46 sequences)

SEQ ID NO: 1 - RNA, 23 nt, Mus musculus
uggagugugacaaugguguuugu

SEQ ID NO: 2 - RNA, 22 nt, Mus musculus
uagcagcacguaaauauuggcg

SEQ ID NO: 3 - RNA, 21 nt, Mus musculus
cugaccuaugaauugacagcc

SEQ ID NO: 4 - RNA, 22 nt, Mus musculus
uguaacagcaacuccaugugga

SEQ ID NO: 5 - RNA, 23 nt, Artificial Sequence
  Description of Artificial Sequence Synthetic oligonucleotide
uggagugugacaaugguguuugu

SEQ ID NO: 6 - RNA, 23 nt, Artificial Sequence
  Description of Artificial Sequence Synthetic oligonucleotide
uggaaugugacaguguugugugu

SEQ ID NO: 7 - RNA, 23 nt, Artificial Sequence
  Description of Artificial Sequence Synthetic oligonucleotide
uggaaugugacaguguugugugu

SEQ ID NO: 8 - RNA, 23 nt, Artificial Sequence
  Description of Artificial Sequence Synthetic oligonucleotide
uggaaugugacaguguugugugu

SEQ ID NO: 9 - RNA, 23 nt, Artificial Sequence
  Description of Artificial Sequence Synthetic oligonucleotide
uggagugugacaaugguguuugu

SEQ ID NO: 10 - RNA, 23 nt, Artificial Sequence
  Description of Artificial Sequence Synthetic oligonucleotide
uggagugugacaaugguguuugu

SEQ ID NO: 11 - RNA, 23 nt, Artificial Sequence
  Description of Artificial Sequence Synthetic oligonucleotide
uggaaugugacaguguugugugu

SEQ ID NO: 12 - RNA, 23 nt, Artificial Sequence
  Description of Artificial Sequence Synthetic oligonucleotide
uggaaugugacaguguugugugu

SEQ ID NO: 13 - DNA, 23 bp, Artificial Sequence
  Description of Artificial Sequence Synthetic oligonucleotide
tggagtgtganaatggtgtttgt

SEQ ID NO: 14 - DNA, 23 bp, Artificial Sequence
  Description of Artificial Sequence Synthetic oligonucleotide
tggagtgtganaatggtgtttgt

SEQ ID NO: 15 - DNA, 23 bp, Artificial Sequence
  Description of Artificial Sequence Synthetic oligonucleotide
tggaatgtganagtgttgtgtgt

SEQ ID NO: 16 - DNA, 23 bp, Artificial Sequence
  Description of Artificial Sequence Synthetic oligonucleotide
tggaatgtganagtgttgtgtgt

SEQ ID NO: 17 - RNA, 22 nt, Artificial Sequence
  Description of Artificial Sequence Synthetic oligonucleotide
uggngugugncnnugguguuug

SEQ ID NO: 18 - RNA, 23 nt, Artificial Sequence
  Description of Artificial Sequence Synthetic oligonucleotide
uggagugugacaaugguguuugu

SEQ ID NO: 19 - RNA, 23 nt, Artificial Sequence
  Description of Artificial Sequence Synthetic oligonucleotide
uggagugugacaaugguguuugu

SEQ ID NO: 20 - RNA, 23 nt, Artificial Sequence
  Description of Artificial Sequence Synthetic oligonucleotide
uggaaggugacaguguuguuugu

SEQ ID NO: 21 - RNA, 23 nt, Artificial Sequence
  Description of Artificial Sequence Synthetic oligonucleotide
uggagugugacaaugguguuugu

SEQ ID NO: 22 - RNA, 23 nt, Artificial Sequence
  Description of Artificial Sequence Synthetic oligonucleotide
uggagugugacaaugguguuugu

SEQ ID NO: 23 - RNA, 23 nt, Artificial Sequence
  Description of Artificial Sequence Synthetic oligonucleotide
uggagugugacaaugguguuugu

SEQ ID NO: 24 - RNA, 23 nt, Artificial Sequence
  Description of Artificial Sequence Synthetic oligonucleotide
uggagugugacaaugguguuugu

SEQ ID NO: 25 - RNA, 23 nt, Artificial Sequence
  Description of Artificial Sequence Synthetic oligonucleotide
uggagugugacaaugguguuugu

SEQ ID NO: 26 - RNA, 23 nt, Artificial Sequence
  Description of Artificial Sequence Synthetic oligonucleotide
uggagugugacaaugguguuugu

SEQ ID NO: 27 - RNA, 23 nt, Artificial Sequence
  Description of Artificial Sequence Synthetic oligonucleotide
uggagugugacaaugguguuugu

SEQ ID NO: 28 - RNA, 23 nt, Artificial Sequence
  Description of Artificial Sequence Synthetic oligonucleotide
uggagugugacaaugguguuugu

SEQ ID NO: 29 - RNA, 23 nt, Artificial Sequence
  Description of Artificial Sequence Synthetic oligonucleotide
uggagugugacaangguguuugu

SEQ ID NO: 30 - RNA, 23 nt, Artificial Sequence
  Description of Artificial Sequence Synthetic oligonucleotide
uggagugngacaaugguguuugu

SEQ ID NO: 31 - RNA, 23 nt, Artificial Sequence
  Description of Artificial Sequence Synthetic oligonucleotide
uggagugngacaangguguuugu

SEQ ID NO: 32 - RNA, 23 nt, Artificial Sequence
  Description of Artificial Sequence Synthetic oligonucleotide
uggagugugacaangguguuugu

SEQ ID NO: 33 - RNA, 23 nt, Artificial Sequence
  Description of Artificial Sequence Synthetic oligonucleotide
uggagugngacaaugguguuugu

SEQ ID NO: 34 - RNA, 23 nt, Artificial Sequence
  Description of Artificial Sequence Synthetic oligonucleotide
uggagugngacaangguguuugu

SEQ ID NO: 35 - RNA, 23 nt, Artificial Sequence
  Description of Artificial Sequence Synthetic oligonucleotide
uggagugugacaangguguuugu

SEQ ID NO: 36 - RNA, 23 nt, Artificial Sequence
  Description of Artificial Sequence Synthetic oligonucleotide
uggagugngacaaugguguuugu

SEQ ID NO: 37 - RNA, 23 nt, Artificial Sequence
  Description of Artificial Sequence Synthetic oligonucleotide
uggagugngacaangguguuugu

SEQ ID NO: 38 - RNA, 23 nt, Artificial Sequence
  Description of Artificial Sequence Synthetic oligonucleotide
uggagugugacaangguguuugu

SEQ ID NO: 39 - RNA, 23 nt, Artificial Sequence
  Description of Artificial Sequence Synthetic oligonucleotide
uggagugngacaaugguguuugu

SEQ ID NO: 40 - RNA, 23 nt, Artificial Sequence
  Description of Artificial Sequence Synthetic oligonucleotide
uggagugngacaangguguuugu

SEQ ID NO: 41 - RNA, 23 nt, Artificial Sequence
  Description of Artificial Sequence Synthetic oligonucleotide
uggagugugacaangguguuugu

SEQ ID NO: 42 - RNA, 23 nt, Artificial Sequence
  Description of Artificial Sequence Synthetic oligonucleotide
uggagugngacaangguguuugu

SEQ ID NO: 43 - RNA, 23 nt, Artificial Sequence
  Description of Artificial Sequence Synthetic oligonucleotide
uggagugugacaangguguuugu

SEQ ID NO: 44 - RNA, 23 nt, Artificial Sequence
  Description of Artificial Sequence Synthetic oligonucleotide
uggagugngacaaugguguuugu

SEQ ID NO: 45 - RNA, 23 nt, Artificial Sequence
  Description of Artificial Sequence Synthetic oligonucleotide
uggagugngacaangguguuugu

SEQ ID NO: 46 - RNA, 23 nt, Artificial Sequence
  Description of Artificial Sequence Synthetic oligonucleotide
uggagugngacaaugguguuugu